(19) United States
(12) Patent Application Publication
(10) Pub. No.: US 2026/0325013 A1
(43) Pub. Date: Oct. 22, 2026

(54) INFORMATION PROCESSING SYSTEM, AND CONTROL METHOD

(71) Applicant: SEKISUI CHEMICAL CO., LTD., Osaka (JP)

(72) Inventors: Yasuyuki SHIRASAKA, Osaka (JP); Akihiro UENISHI, Ibaraki (JP); Jiyunichi MATSUZAKI, Ibaraki (JP); Tetsuya ISHII, Ibaraki (JP)

(21) Appl. No.: 19/110,622

(22) PCT Filed: Sep. 21, 2023
(86) PCT No.: PCT/JP2023/034228

§ 371 Date: Mar. 11, 2025

(30) Foreign Application Priority Data
Sep. 29, 2022 (JP) 2022-157010

(51) Int. Cl. A61B 5/113; A61B 5/00; A61B 7/00

(52) U.S. Cl. CPC A61B 5/113; A61B 5/6804; A61B 5/6823; A61B 5/6843; A61B 5/6891; A61B 5/725; A61B 7/003

(57) ABSTRACT

An information processing system (100) extracts a pressure signal and a breathing signal from a detection signal outputted from a sensor (11) for detecting vibrations emitted from the trunk of a subject, determines, based on a fluctuation in the strength of the pressure signal, a breathing phase of the subject, and determines, based on the breathing signal, whether a sound associated with the breathing contains an abnormal sound. In a case where the sound associated with the breathing contains an abnormal sound, the information processing system (100) estimates in which breathing phase the abnormal sound is generated, an exhalation segment or an inhalation segment.

### TECHNICAL FIELD

The present disclosure relates to an information processing system, etc. for detecting vibrations emitted by a subject.

### BACKGROUND ART

Various methods have been developed for measuring vibrations (including: a sound associated with breathing; heartbeats; etc.) emitted by a subject. For example, Patent Literature 1 discloses a technique for non-invasively monitoring at least one breathing pattern, at least one heartbeat pattern, or both of a subject who is sleeping.

### CITATION LIST

### Patent Literature

### Patent Literature 1

Japanese Patent Application Publication Tokukai No. 2009-501557

### SUMMARY OF INVENTION

### Technical Problem

The types of abnormal sounds (including adventitious sounds such as fine crackles and coarse crackles) contained in sounds associated with the breathing of a subject provide information regarding a disease the subject has. For identifying the type of abnormal sound, in which of the exhalation and inhalation segments of the breathing of the subject the abnormal sound is generated is important in some cases.

### Solution to Problem

In order for the above problem to be solved, an information processing system in accordance with an aspect of the present disclosure includes: a sensor for detecting, at a position where the sensor does not touch a subject or a position where the sensor touches the subject on a trunk of the subject, vibrations emitted from the trunk; a signal extracting section for extracting a pressure signal and a breathing signal from a detection signal outputted from the sensor, the pressure signal indicating a pressure on the sensor, the breathing signal indicating a sound associated with breathing of the subject; a breathing phase determining section for determining, based on a fluctuation in a strength of the pressure signal, an exhalation segment and an inhalation segment of the subject; an abnormal sound determining section for determining, based on the breathing signal, whether the sound associated with the breathing contains an abnormal sound; and an estimating section for estimating, in a case where the sound associated with the breathing contains an abnormal sound, in which segment the abnormal sound is generated, the exhalation segment or the inhalation segment.

### Advantageous Effects of Invention

With an aspect of the present disclosure, it is possible to accurately estimate a breathing phase (exhalation segment or inhalation segment) in which an abnormal sound contained in a sound associated with the breathing of a subject is generated.

### BRIEF DESCRIPTION OF DRAWINGS

FIG. 1 is a conceptual diagram illustrating an example configuration of an information processing system in accordance with an embodiment of the present invention.

FIG. 2 is a diagram illustrating an example schematic configuration of an information processing apparatus of the information processing system.

FIG. 3 is a diagram illustrating another example schematic configuration of the information processing apparatus of the information processing system.

FIG. 4 is a diagram illustrating still another example schematic configuration of the information processing apparatus of the information processing system.

FIG. 5 is a functional block diagram illustrating an example configuration of the information processing system.

FIG. 6 is a representation for explaining various signals contained in a detection signal.

FIG. 7 is a diagram for explaining the classifications of adventitious sounds included in an abnormal sound and representative cases corresponding to the respective adventitious sounds.

FIG. 8 is a flowchart illustrating an example flow of processes carried out by the components of the information processing system.

FIG. 9 is a representation of an example of a display screen displayed on a display section of a communication apparatus of the information processing system.

FIG. 10 is a functional block diagram illustrating a variation of the configuration of the information processing system.

FIG. 11 is a functional block diagram illustrating an example configuration of an information processing system in accordance with Embodiment 2 of the present invention.

FIG. 12 is a flowchart illustrating an example flow of processes carried out by the information processing system.

FIG. 13 is a representation of an example of a display screen displayed on a display section of a communication apparatus of the information processing system.

FIG. 14 is a functional block diagram illustrating a variation of the configuration of the information processing system.

FIG. 15 is a functional block diagram illustrating another variation of the configuration of the information processing system.

FIG. 16 is a flowchart illustrating a flow of processes carried out by an abnormal sound determining section of an information processing system in accordance with Embodiment 3 of the present invention.

FIG. 17 is a representation of graphs illustrating a specific example of the processes carried out by the abnormal sound determining section of the information processing system in accordance with Embodiment 3 of the present invention.

FIG. 18 is a representation of graphs illustrating results of the processes carried out by the abnormal sound determining section of the information processing system in accordance with Embodiment 3 of the present invention.

FIG. 19 is a representation of graphs illustrating results of the processes carried out by the abnormal sound determining section of the information processing system in accordance with Embodiment 3 of the present invention.

FIG. 20 is a representation of a specific example of the processes carried out by the abnormal sound determining section of the information processing system in accordance with Embodiment 3 of the present invention.

FIG. 21 is a representation of a specific example of the processes carried out by the abnormal sound determining section of the information processing system in accordance with Embodiment 3 of the present invention.

FIG. 22 is a representation of a specific example of the processes carried out by the abnormal sound determining section of the information processing system in accordance with Embodiment 3 of the present invention.

FIG. 23 is a graph illustrating results of the processes carried out by the abnormal sound determining section of the information processing system in accordance with Embodiment 3 of the present invention.

### DESCRIPTION OF EMBODIMENTS

### Embodiment 1

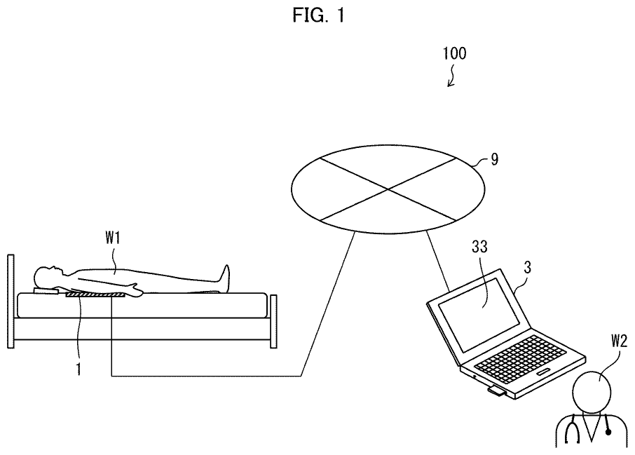

The following description will discuss embodiments of the present disclosure in detail with reference to FIGS. 1 to 10.

### Outline of Information Processing System 100

An information processing system 100 in accordance with Embodiment 1 of the present disclosure is a system for, based on a detection signal outputted from a sensor for detecting, at a position where the sensor does not touch a subject W1 or a position where the sensor touches the subject W1 on the trunk, vibrations emitted from the trunk of the subject W1, estimating in which breathing phase an abnormal sound contained in a sound associated with the breathing of the subject W1 is generated, the exhalation segment or the inhalation segment.

In this specification, the “subject” is typically a person who needs to be monitored by a medical expert W2 or the like, such as a patient lying on a bed or the like. The “detection signal” is a signal that indicates vibrations emitted by the subject W1, and is raw data outputted from the sensor or data obtained by subjecting the raw data to an amplification process or a noise removal process. The noise removal process can be carried out by, for example, performing a filtering process on the raw data with respect to a region of not less than 2,000 Hz. The “trunk” of the subject W1 is typically at least one of body parts of the subject W1 which are the chest, the abdomen, and the back, but may be other than these body parts.

### Configuration of Information Processing System 100

First, the configuration of the information processing system 100 is described here with use of FIG. 1. FIG. 1 is a conceptual diagram of an example configuration of the information processing system 100.

As illustrated in FIG. 1, the information processing system 100 may include an information processing apparatus 1 and a communication apparatus 3. The numbers of information processing apparatuses 1 and communication apparatuses 3 included in the information processing system 100 may each be one, or may each be more than one.

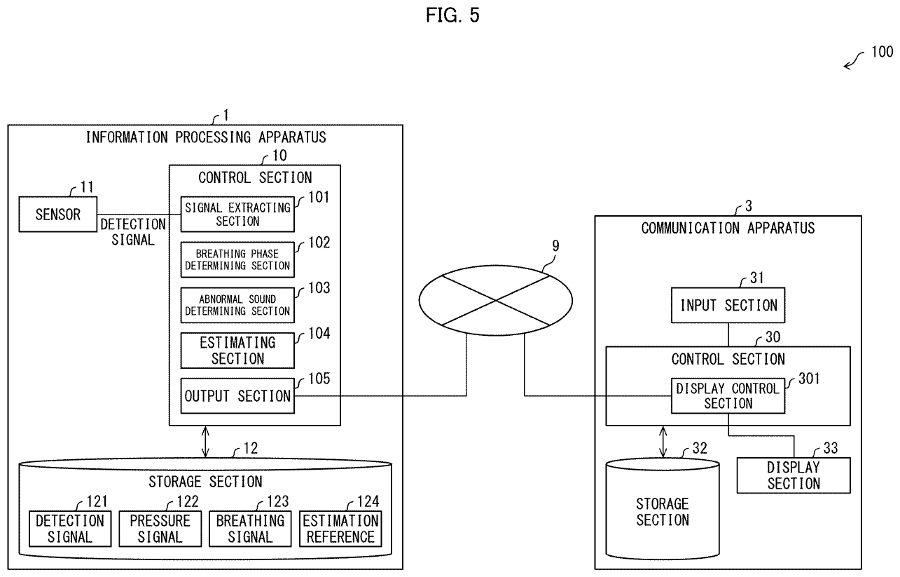

The information processing apparatus 1 analyzes a detection signal which is outputted from a sensor 11 (see FIG. 5) for detecting, at a position where the sensor 11 does not touch the subject W1 or a position where the sensor 11 touches the subject W1 on the trunk, vibrations emitted from the trunk of the subject W1 and which indicates the vibrations emitted from the trunk of the subject W1, to estimate, in a case where a sound associated with the breathing of the subject W1 contains an abnormal sound, in which breathing phase the abnormal sound is generated, the exhalation segment or the inhalation segment, and outputs the result of the estimation to the outside.

The information processing apparatus 1 may output, to the outside, information regarding the frequency characteristic of an abnormal sound signal which indicates an abnormal sound contained in the breathing signal extracted from the detection signal, together with the result of the estimation. Further, the information processing apparatus 1 may output, to the outside, an estimation result obtained by estimating what adventitious sound type the abnormal sound is. Furthermore, the information processing apparatus 1 may output, to the outside, information regarding a health condition corresponding to the estimated adventitious sound type. Typically, the location to which the result of the estimation and others are outputted is the communication apparatus 3. Typically, the communication apparatus 3 is a computer, a smartphone, a tablet terminal, or the like used by the medical expert W2 or the like, and is installed in, for example, nurses station.

The information processing apparatus 1 may further estimate the type of adventitious sound included in the abnormal sound contained in the breathing signal and output, to the outside (e.g. communication apparatus 3), the result of the estimation of the adventitious sound type.

The sensor 11 is installed at a position where the sensor 11 does not touch the subject W1 or a position where the sensor 11 touches the subject W1 on the trunk. The sensor 11 may be installed on a bed on which the subject W1 is lying. The position where the sensor 11 is installed may be between the bed and a mattress, under the mattress, or may be between a bed sheet and the mattress, above the mattress. In a case where the subject W1 is wearing clothes, the position where the sensor 11 is installed may be the top surface of the bed. In installing the sensor 11 at any of these positions, the sensor 11 is preferably typically formed in the shape of a thin plate (sheet). This makes it possible to place the sensor 11 at various locations.

As above, since the information processing system 100 uses the sensor 11 installed at a position where the sensor 11 does not touch the subject W1 or a position where the sensor 11 touches the subject W1 on the trunk, the subject W1 does not need to be in a predetermined detection area or to assume a predetermined position for detection.

The information processing apparatus 1 and the communication apparatus 3 may be directly connected together, or may be connected together so as to be capable of communicating with each other over a communication network 9, as illustrated in FIG. 1. The communication network 9 is not limited to any form, but may be a local area network (LAN) or may be the Internet.

The information processing system 100 may include, in addition to the information processing apparatus 1 and the communication apparatus 3, a server (not illustrated) communicably connected to the information processing apparatus 1 and the communication apparatus 3. For example, the server may have a configuration in which estimation results transmitted from a plurality of information processing apparatuses 1 are stored and managed for each of the subjects W1. The medical expert W2 or the like may be able to make access to the server via the communication apparatus 3, to refer to the estimation result regarding each subject W1.

### Configuration of Information Processing Apparatus 1

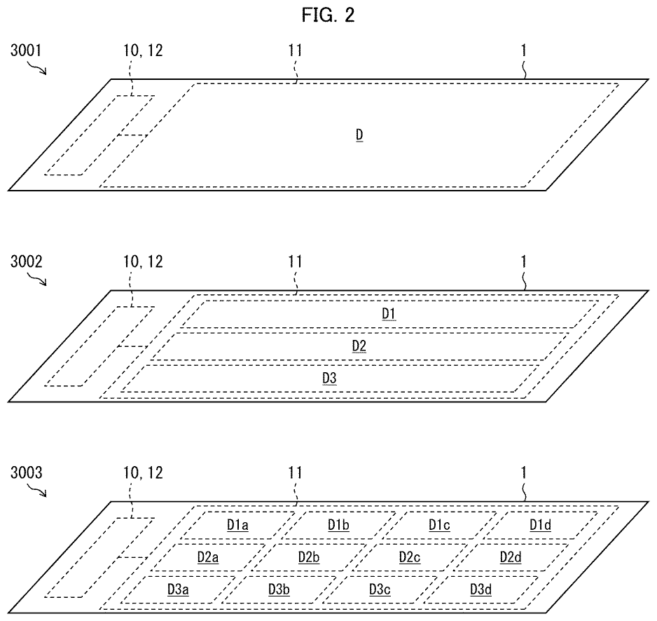

Next, the appearance and schematic configuration of the information processing apparatus 1 are described here with reference of FIGS. 2 to 4 and FIG. 6 and with use of FIG. 5. FIG. 5 is a functional block diagram illustrating an example configuration of the information processing apparatus 1.

As illustrated in FIG. 5, the information processing apparatus 1 includes the sensor 11, a control section 10, and a storage section 12.

### Sensor 11

The sensor 11 is a sensor capable of detecting, at a position where the sensor 11 does not touch the subject W1 or a position where the sensor 11 touches the subject W1 on the trunk, vibrations emitted from the trunk of the subject W1. The sensor 11 is not limited to a particular type. For example, the sensor 11 is a piezoelectric sensor. Adopting a piezoelectric sensor as the sensor 11 facilitates thickness reduction and therefore makes it possible to reduce the possibility of making the subject feel uncomfortable. Examples of the piezoelectric sensor include a piezoelectric sensor which develops current upon compressional deformation, a piezoelectric sensor which develops current upon extensional deformation, and a piezoelectric sensor which develops current upon torsional deformation. In a case where more accurate detection is desired, it is preferable to use, as the sensor 11, a piezoelectric sensor which develops current upon compressional deformation. In a case where more accurate detection of signals of low frequencies is desired, it is preferable to use, as the sensor 11, a piezoelectric sensor which includes foam.

The sensor 11 is preferably capable of detecting vibrations of a wide frequency band. This eliminates the need for disposing different types of sensors for different frequency bands, makes it easy for the medical expert W2 to carry out maintenance and management, and therefore improves convenience.

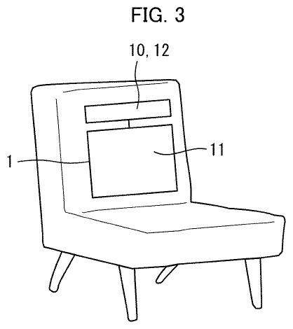

FIGS. 2 to 4 are diagrams illustrating schematic configurations of the information processing apparatus 1. As illustrated in FIG. 1, the sensor 11 may be installed on the bed on which the subject W1 is lying, at a position at which the trunk is supported. In a case of installing the sensor 11 on the bed, the sensor 11 may be preferably formed in the shape of a thin plate (sheet), as illustrated in FIG. 2. Alternatively, the sensor 11 may be installed on a chair on which the subject W1 sits, at a position at which the trunk is supported, as illustrated in FIG. 3. This enables the sensor 11 to detect vibrations emitted from the trunk of the subject W1 who is lying on a bed or sitting on a chair and is therefore in a natural posture.

In a case of installing the sensor 11 on a bed, the sensor 11 may be installed between a bed sheet and a mattress, above the mattress. Further, in a case where the subject W1 is wearing clothes, the sensor 11 may be installed at the top surface of the bed.

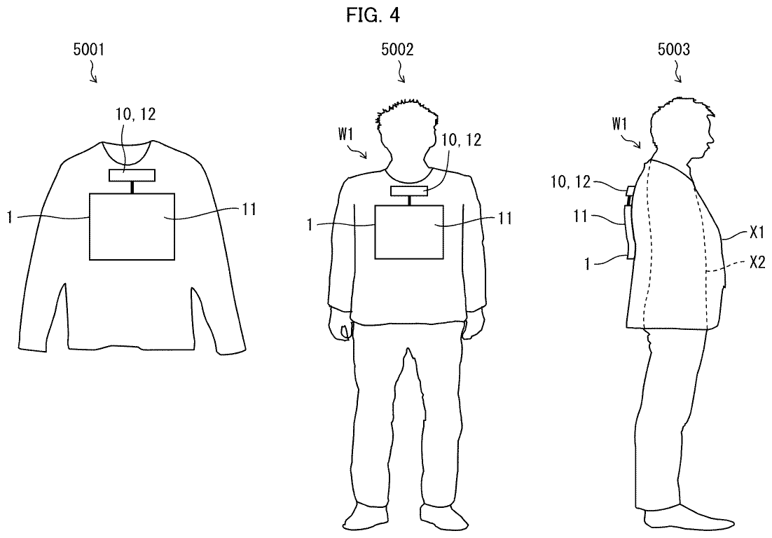

In addition, the sensor 11 may be installed on a piece of clothing the subject W1 is wearing, as illustrated in FIG. 4. The reference sign 5001 of FIG. 4 illustrates an example installation of the sensor 11 on a piece of clothing. The reference signs 5002 and 5003 of FIG. 4 illustrate examples in which the subject W1 is wearing a piece of clothing on which the sensor 11 is installed. As illustrated in 5001 of FIG. 4, the sensor 11 may be installed on a piece of clothing so as to be located on the chest side of the subject W1 when the subject W1 wears the piece of clothing. Further, as illustrated in 5003 of FIG. 4, the sensor 11 may be installed on a piece of clothing so as to be located on the back side of the subject W1 when the subject W1 wears the piece of clothing.

In a case of installing the sensor 11 of the information processing apparatus 1 on a piece of clothing the subject W1 is wearing, the sensor 11 may be installed on the piece of clothing (reference sign X1 of FIG. 4) the subject W1 is wearing, at a position where the piece of clothing is sandwiched between the sensor 11 and the trunk (reference sign X2 of FIG. 4), as illustrated in 5003 of FIG. 4. In this case, it is possible to detect vibrations emitted by the subject W1 without causing the sensor 11 to touch the subject W1. In addition, with this configuration, even in a case where the subject W1 moves from one location to another, as long as the subject W1 is wearing the piece of clothing including the sensor 11, it is possible to detect vibrations emitted from the trunk of the subject W1.

The sensor 11 may include one or more detection areas. In a case where the sensor 11 includes a plurality of detection areas, the sensor 11 may output respective detection signals detected in the plurality of detection areas. In a case where the sensor 11 is formed in the shape of a thin plate, the plurality of detection areas may be disposed side by side in the same plane. For example, the information processing apparatus 1 illustrated in 3001 of FIG. 2 includes the sensor 11 which includes a single detection area D. The information processing apparatus 1 illustrated in 3002 of FIG. 2 includes the sensor 11 which includes detection areas D1 to D3 arranged in three columns. The information processing apparatus 1 illustrated in 3003 of FIG. 2 includes the sensor 11 which includes detection areas D1a to D3d arranged in four rows and three columns.

For example, in a case of the information processing apparatus 1 illustrated in 3002 of FIG. 2, detection signals detected in the respective detection areas D1 to D3 are individually outputted. Similarly, in a case of the information processing apparatus 1 illustrated in 3003 of FIG. 3, detection signals detected in the respective detection areas D1a to D3d are individually outputted. Each of the detection areas D1a to D3d may be, for example, 10 cm square.

By adopting the configuration in which a plurality of detection areas are included, it is possible to correctly identify the site at which an abnormal sound in the breathing signal of the subject W1 is generated, by individually analyzing the detection signals from the respective detection areas and making mutual comparisons between the results of the analysis.

### Control Section 10 and Storage Section 12

As an example, the control section 10 may be a central processing unit (CPU). The control section 10 retrieves a control program which is software stored in the storage section 12, and loads the control program into a memory such as a random access memory (RAM), to implement various functions. As illustrated in FIG. 5, the control section 10 includes a signal extracting section 101, a breathing phase determining section 102, an abnormal sound determining section 103, an estimating section 104, and an output section 105. It should be noted that for the sake of simplicity of description, the illustration of the control program is omitted in the storage section 12 illustrated in FIG. 5.

The signal extracting section 101 obtains the detection signal outputted from the sensor 11, and extracts, from the obtained detection signal, a pressure signal indicating a pressure on the sensor 11 and a breathing signal indicating a sound associated with the breathing of the subject W1.

The sensor 11 can detect vibrations which are emitted from the subject W1 and which have many different frequency regions. Thus, the detection signal outputted from the sensor 11 is a signal in which a plurality of vibrations having different frequency characteristics are superposed on each other. As an example, the sensor 11 may be capable of obtaining a pressure signal having a frequency of not less than 0.1 Hz and not more than 1 Hz and a breathing signal having a frequency of not less than 20 Hz and not more than 1,000 Hz. The pressure signal and the breathing signal are distinguishable from each other on the basis of the frequency components (spectrum).

As above, the information processing apparatus 1 may include the sensor 11 capable of detecting vibrations of a wide frequency band. This eliminates the need for disposing a plurality of different types of sensors in the information processing apparatus 1, makes it easy for the medical expert W2 or the like who uses the information processing system 100 to carry out maintenance and management of the information processing apparatus 1, and therefore improves the convenience of the information processing apparatus 1.

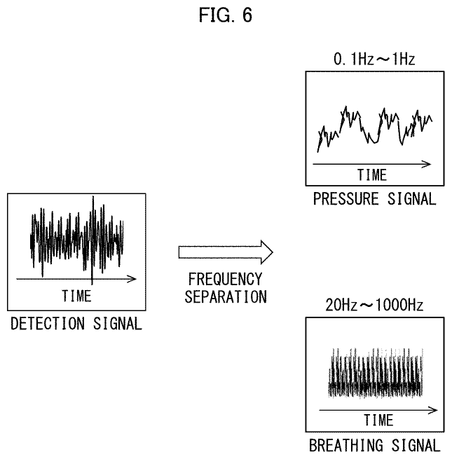

The signal extracting section 101 may apply a well-known approach for frequency separation or the like, to extract the pressure signal and the breathing signal from the detection signal. This is described here with use of FIG. 6. FIG. 6 is a representation for explaining various signals contained in the detection signal. As illustrated in FIG. 6, by separating the frequencies, it is possible to extract, from the detection signal, a pressure signal and a breathing signal which have frequency characteristics different from each other.

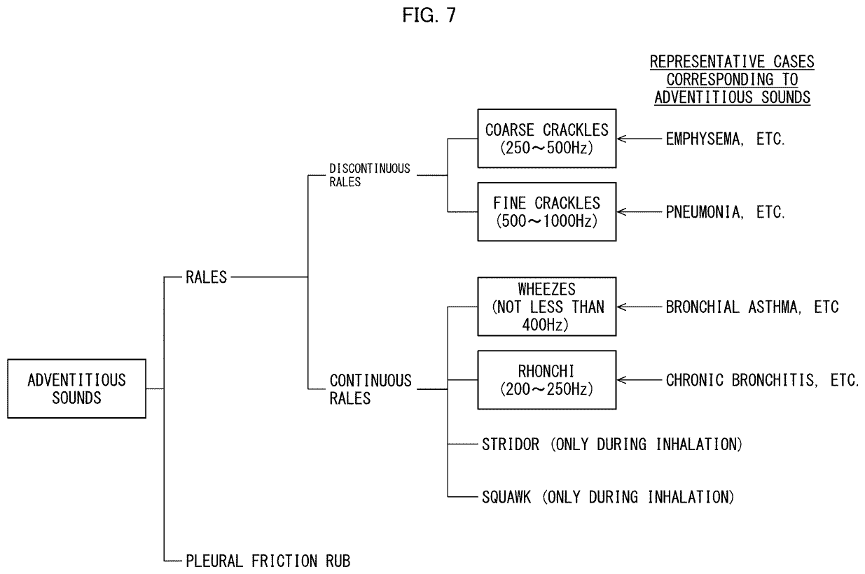

The signal extracting section 101 may use band-pass filters corresponding to respective frequencies of the adventitious sound types, to extract a breathing signal from the detection signal. As will be described later, the frequencies of respective adventitious sound types are described in FIG. 7. For example, in a case of extracting coarse crackles from the detection signal, a band-pass filter corresponding to 250 Hz to 500 Hz may be used for the extraction.

The signal extracting section 101 stores, in the storage section 12, the detection signal obtained from the sensor 11, and the pressure signal and the breathing signal extracted from the detection signal (detection signal 121, pressure signal 122, and breathing signal 123). These signals may be stored together with time information which indicates the time at which the detection signal, which is the source of the extraction, is detected.

The breathing phase determining section 102 determines, based on the fluctuation of the strength of the pressure signal extracted in the signal extracting section 101, an exhalation segment and an inhalation segment of the subject. For example, by taking, as the inhalation segment, a segment in which the strength of the pressure signal exhibits an increasing tendency and taking, as the exhalation segment, a segment in which the strength of the pressure signal exhibits a decreasing tendency, the breathing phase may be determined.

The abnormal sound determining section 103 determines, based on the breathing signal extracted in the signal extracting section 101, whether a sound associated with breathing contains an abnormal sound. The abnormal sound determining section 103 may determine, in at least one of cases (A1) to (A4) below, that a sound associated with breathing contains an abnormal sound.

(A1) The breathing signal has a predetermined frequency characteristic which indicates an abnormal sound.

(A2) A signal having a strength greater than a predetermined strength is contained for each of the frequency bands of the breathing signal.

(A3) A signal having a strength greater than a predetermined strength is contained in a frequency band, detected during abnormal breathing, of the breathing signal.

(A4) The breathing signal has a ratio between the strength of a signal in a frequency band detected during normal breathing and the strength of a signal in a frequency band detected during abnormal breathing, the ratio being equal to or greater than a predetermined threshold.

The “predetermined frequency characteristic” in (A1) above is any of the different frequency characteristics of various adventitious sounds. For example, as illustrated in FIG. 7, the frequency characteristic of the coarse crackles is 250 Hz to 500 Hz, the frequency characteristic of the fine crackles is 500 Hz to 1,000 Hz, the frequency characteristic of the wheezes is not less than 400 Hz, and the frequency characteristic of the rhonchi is 200 Hz to 250 Hz. In a case where the breathing signal has any of these frequency characteristics, it may be determined that the sound associated with breathing contains an abnormal sound.

The “predetermined strength . . . for each of the frequency bands” in (A2) above may be, for example, the strength of a signal of around 100 Hz in the state where a normal breath sound of less than 200 Hz is obtained.

The “frequency band detected during abnormal breathing” in (A3) above may be, for example, around 200 Hz in the state where a normal breath sound of less than 200 Hz is obtained. The “predetermined strength” may be the strength of a signal of around 100 Hz in the state where a normal breath sound of less than 200 Hz is obtained.

The “predetermined threshold” in (A4) above is, for example, the strength of a signal of around 100 Hz in the state where a normal breath sound of less than 200 Hz is obtained. In a case where a signal strength equivalent to or greater than the predetermined threshold is detected around 200 Hz, it may be determined that the sound associated with breathing contains an abnormal sound.

The estimating section 104 estimates, based on the determination result provided by the breathing phase determining section 102 or the determination result provided by the abnormal sound determining section 103, an adventitious sound type. The estimation of an adventitious sound type in the estimating section 104 is made based on at least one selected from the group consisting of (i) in which segment an abnormal sound is contained, the exhalation segment or the inhalation segment, (ii) the frequency of an abnormal sound signal indicating the abnormal sound, (iii) a change in sound pressure of the abnormal sound in the exhalation segment or the inhalation segment, and (iv) the time interval between occurrences of the abnormal sound in the exhalation segment or the inhalation segment. Such an estimation is made with reference to an estimation reference 124 stored in the storage section 12.

The estimation reference 124 contains information on the characteristics (generation segment, frequency characteristic, change in sound pressure, time interval, etc.) of various adventitious sounds.

Furthermore, the estimating section 104 may calculate a smoothed strength obtained by smoothing the fluctuation with time in the strengths of signals in the same frequency band which contains the frequency of the abnormal sound signal, to estimate the adventitious sound type in accordance with the smoothed strength.

As an example of the smoothing process, summation averaging is performed in the present embodiment. With summation averaging, by adding up and averaging many pieces of measured data, it is possible to cancel out and reduce noise contained in a signal. In the present embodiment, a smoothed strength may be found which is obtained by performing the summation averaging of the strengths of signals with the passage of measurement time, the signals being in the same frequency band which contains the frequency of the abnormal sound signal. Other examples of the smoothing process include moving averaging. With these processes, it is possible to estimate an adventitious sound type with high accuracy and high sensitivity.

The output section 105 may output, to the communication apparatus 3, information regarding an estimation result in the estimating section 104 and a health condition based on the estimation result. The outputted information regarding a health condition may be information obtained via the communication network 9, or may be information stored in advance in the storage section 12.

### Configuration of Communication Apparatus 3

Next, the configuration of the communication apparatus 3 is described here with use of FIG. 5. The communication apparatus 3 includes an input section 31, a control section 30, a storage section 32, and a display section 33.

The input section 31 may be a keyboard, a touch panel, a mouse, or the like. The control section 30 retrieves a control program which is software stored in the storage section 32, and loads the control program into a memory such as a RAM, to implement various functions. The control section 30 includes a display control section 301 for displaying various pieces of information on the display section 33.

### Correspondence between Adventitious Sound Included in Abnormal Sound and Disease

The reason why estimating the type of adventitious sound contained in a sound associated with the breathing of the subject W1 with high accuracy is important to obtain correct information regarding the health condition of the subject W1 is described here, by citing a specific example. FIG. 7 is a diagram for explaining the classifications of adventitious sounds and representative cases corresponding to the respective adventitious sounds.

An adventitious sound is a sound included in an abnormal sound in a sound associated with breathing. The adventitious sounds include “rales”, “pleural friction rub”, etc. The “rales” include discontinuous rales and continuous rales. The discontinuous rales contain coarse crackles and fine crackles, and the continuous rales contain wheezes, rhonchi, stridor, and squawk. That is, the adventitious sound contains at least one selected from the group consisting of the coarse crackles, fine crackles, wheezes, rhonchi, stridor, squawk, and pleural friction rub of the subject W1.

Both the coarse crackles and the fine crackles have the characteristics of being discontinuous and generated in the inhalation segment. The rhonchi have the characteristics of being continuous and generated in both the exhalation segment and the inhalation segment. The wheezes have the characteristics of being continuous and generated in the exhalation segment.

In a case where coarse crackles are mixed in a sound associated with the breathing of the subject W1, the subject W1 can have pneumonia, alveolar hemorrhage, heart failure, emphysema, etc., and in a case where fine crackles are mixed, the subject W1 can have pneumonia, etc. In a case where wheezes are mixed in a sound associated with the breathing of the subject W1, the subject can have chronic obstructive pulmonary disease (COPD), bronchial asthma, etc. and in a case where rhonchi are mixed, the subject W1 can have chronic bronchitis, etc.

Thus, when it is possible to estimate an adventitious sound type based on a sound associated with the breathing of the subject W1, it is possible to estimate and early find a disease the subject W1 can have. However, in order to accurately diagnose the health condition of the subject W1, it is necessary to, based on the pressure signal and the breathing signal, first accurately estimate the exhalation segment and the inhalation segment of the subject W1 and estimate in which breathing phase the abnormal sound is generated. When it is possible to estimate the breathing phase in which the abnormal sound is generated, it is possible to accurately estimate the type of adventitious sound contained in a sound associated with the breathing of the subject W1. By adopting the information processing system 100 of the present disclosure, it is possible to, based on the pressure signal and the breathing signal of the subject W1, accurately estimate the exhalation segment and the inhalation segment of the subject W1 and accurately estimate what adventitious sound type an abnormal sound contained in a sound associated with the breathing of the subject W1 is. This enables a medical expert or the like to diagnose the health condition of a subject with high accuracy.

### Processes Carried Out by Information Processing System 100

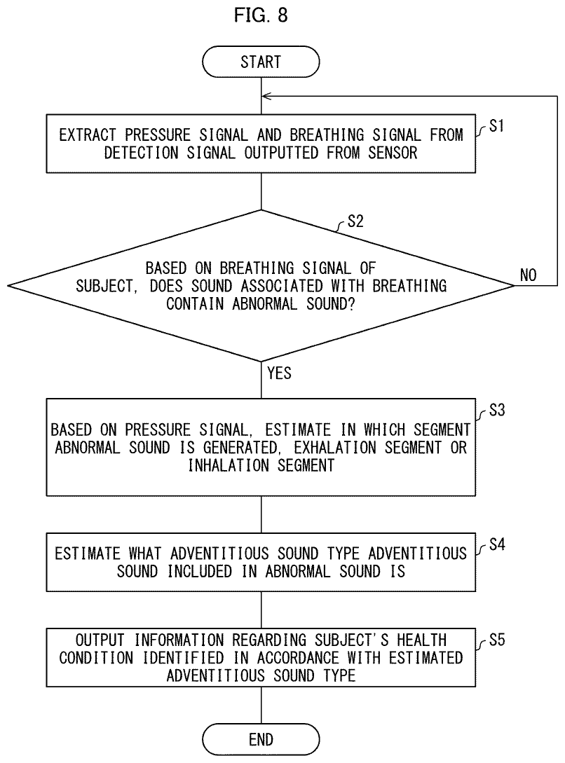

Next, a flow of processes carried out by the information processing system 100 is described here with use of FIG. 8. FIG. 8 is a flowchart illustrating an example flow of processes carried out by the information processing system 100 (e.g. information processing apparatus 1).

First, the signal extracting section 101 extracts a pressure signal and a breathing signal from a detection signal outputted from the sensor 11 (step S1).

Next, the abnormal sound determining section 103 determines, based on the breathing signal extracted in the signal extracting section 101, whether a sound associated with breathing contains an abnormal sound (step S2). In a case of the determination that the sound associated with breathing does not contain an abnormal sound (NO in step S2), the processing returns to step S1.

In a case of the determination that the sound associated with breathing contains an abnormal sound (YES in step S2), the estimating section 104 estimates, based on the pressure signal, in which segment the abnormal sound is generated, the exhalation segment or the inhalation segment (step S3).

The estimating section 104 further estimates, based on the breathing phase determining section 102, the abnormal sound determining section 103, and the estimation reference 124 stored in the storage section 12, what adventitious sound type the adventitious sound included in the abnormal sound is (step S4).

The output section 105 (health condition output section) outputs the adventitious sound type estimated in the estimating section 104 and information regarding the health condition of the subject, the health condition being identified in accordance with the adventitious sound type estimated in the estimating section 104 (step S5).

The timings at which the information processing system 100 carries out the respective processes illustrated in FIG. 8 can be set at any timings. For example, the information processing system 100 may carry out the respective processes illustrated in FIG. 8 every predetermined period (e.g. one hour), or may carry out the respective processes illustrated in FIG. 8 each time it is determined via the sensor 11 that the subject has got out of a bed.

### Example Display

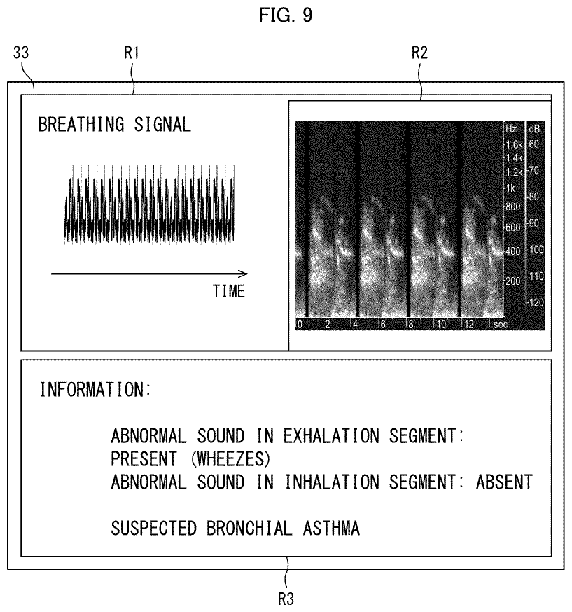

A screen display indicated on the display section 33 in a case where a sound associated with the breathing of the subject W1 contains an abnormal sound and the information processing apparatus 1 obtains (B1) to (B5) below from the information processing apparatus 1 is described here with use of FIG. 9.

(B1) An estimation result obtained by estimating in which breathing phase the abnormal sound is generated, the exhalation segment or the inhalation segment.

(B2) The breathing signal extracted from the detection signal.

(B3) The frequency characteristic of an abnormal sound signal indicating the abnormal sound.

(B4) An estimation result obtained by estimating an adventitious sound type.

(B5) Information regarding a health condition.

FIG. 9 is a representation of an example of a display screen displayed on the display section £ 33 of the communication apparatus 3 of the information processing system 100. As illustrated in FIG. 9, on the display section 33, a region R1 in which to display waveform data of the breathing signal of the subject W1, a region R2 in which to display a sonograph which indicates the frequency characteristic and the strength of an abnormal sound signal indicating an abnormal sound, and a region R3 in which to display the estimation results of (B1) and (B4) and the information of (B5) may be displayed. In the region R3 illustrated in FIG. 9, “Abnormal sound in exhalation segment: present (wheezes)” and “Abnormal sound in inhalation segment: absent”, which are the estimation results of (B1) and (B4), and “Suspected bronchial asthma”, which is the information of (B5), are displayed.

### Variation of Configuration of Information Processing System 100

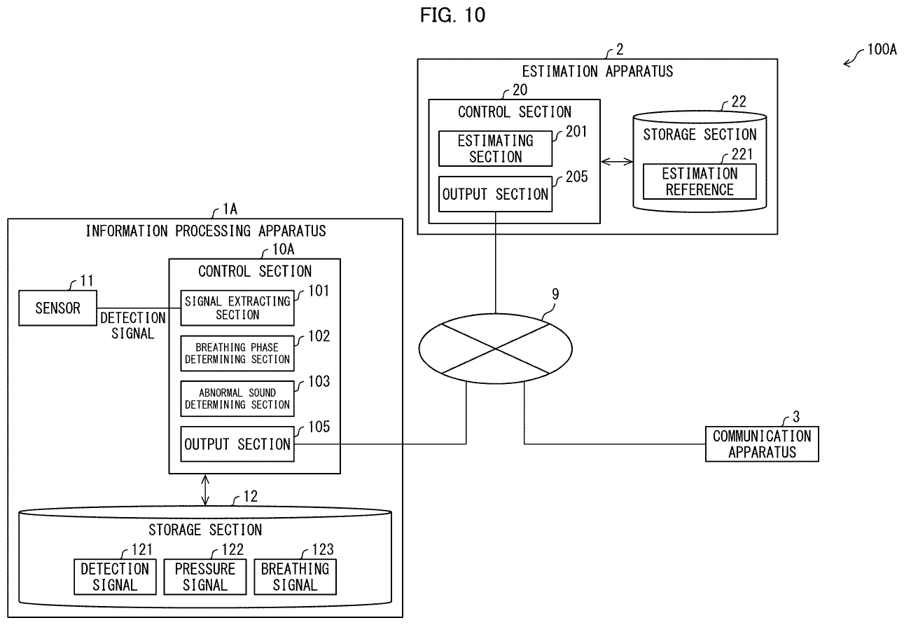

FIG. 10 is a functional block diagram illustrating a variation of the configuration of the information processing system 100.

Compared with the information processing system 100 illustrated in FIG. 5, an information processing system 100A illustrated in FIG. 10 differs from the information processing system 100 in that the estimating section 104 included in the control section 10 of the information processing apparatus 1 is included as an estimating section 201 in an estimation apparatus 2 which is an external apparatus, not in a control section 10A of an information processing apparatus 1A. The estimation apparatus 2 includes a control section 20 and a storage section 22. Further, the control section 20 includes the estimating section 201 and an output section 205.

In accordance with the above variation, the output section 105 of the information processing apparatus 1A outputs determination results provided by the breathing phase determining section 102 and the abnormal sound determining section 103 via the communication network 9 to the estimation apparatus 2.

The estimating section 201 refers to determination results outputted by the output section 105 and an estimation reference 221 stored in the storage section 22, to estimate an adventitious sound type and estimate information regarding the health condition of the subject W1.

The output section 205 outputs the adventitious sound type and the information regarding the health condition of the subject W1 that are estimated in the estimating section 201, to the communication apparatus 3 via the communication network 9.

The configuration of the information processing system 100 is not limited. The configuration in which the sections of the information processing apparatus 1 are newly provided as external apparatuses may be adopted as described above, or the configuration in which the sections of the information processing apparatus 1 are integrated in a single apparatus may be adopted.

### Embodiment 2

The following description will discuss another embodiment of the present disclosure. For convenience of description, a member having the same function as a member described in the above embodiment is assigned the same reference sign, and the description thereof is not repeated here.

### Configuration of Information Processing System 100B

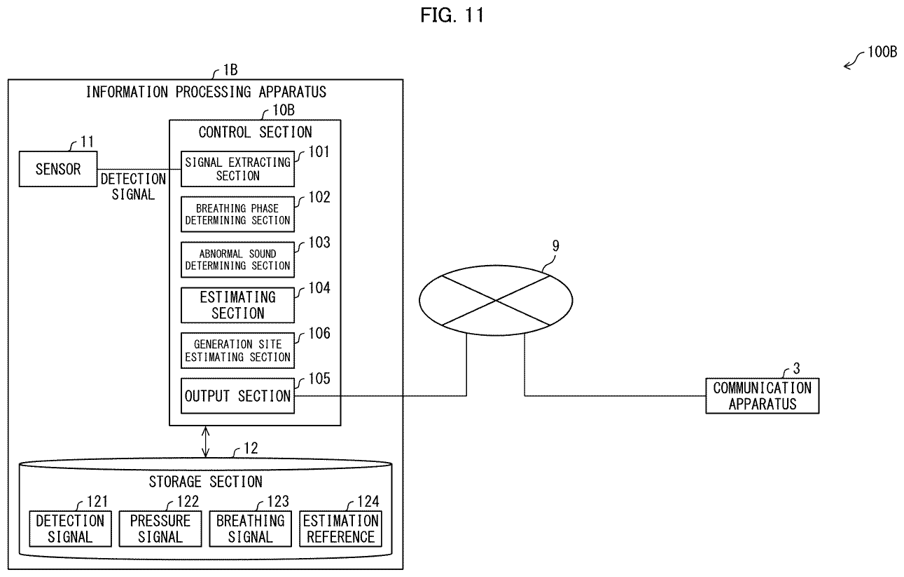

An information processing system 100B is described here with use of FIG. 11. In the information processing system 100B, the configuration in which a sensor 11 includes a plurality of detection areas, and detection signals (hereinafter, area-specific detection signals) detected in the plurality of respective detection areas are analyzed is adopted. FIG. 11 is a functional block diagram illustrating an example of the configuration of the information processing system 100B.

As illustrated in FIG. 11, the information processing system 100B may include an information processing apparatus 1B and a communication apparatus 3. The information processing apparatus 1B includes the sensor 11 which includes a plurality of detection areas, a control section 10B, and a storage section 12. The control section 10B includes a generation site estimating section 106, in addition to a signal extracting section 101, a breathing phase determining section 102, an abnormal sound determining section 103, an estimating section 104, and an output section 105.

The generation site estimating section 106 estimates, based on breathing signals extracted from the respective area-specific detection signals, an abnormal sound generation site inside the subject W1.

The example cited here for description is the configuration in which the information processing apparatus 1B includes the generation site estimating section 106. However, the present disclosure is not limited thereto. For example, the generation site estimating section 106 may be included in the communication apparatus 3 or a server (not illustrated).

### Processes Carried Out by Information Processing System 100B

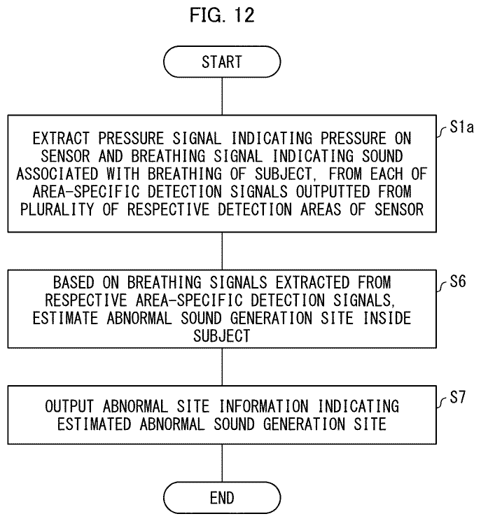

Next, a flow of processes carried out by the information processing system 100B is described here with use of FIG. 12. FIG. 12 is a flowchart illustrating an example flow of processes carried out by the information processing system 100B.

The signal extracting section 101 obtains area-specific detection signals outputted from the respective detection areas (e.g. the detection areas D1 to D4 illustrated in FIG. 3), to extract a pressure signal and a breathing signal from each of the area-specific detection signals (step S1a).

Subsequently, the generation site estimating section 106 estimates, based on the breathing signals extracted from the respective area-specific detection signals, an abnormal sound generation site inside the subject W1 (step S6). For example, the generation site estimating section 106 may identify the position of the detection area having outputted an area-specific detection signal from which a breathing signal containing the greatest number of abnormal sounds is extracted, and estimate, as the abnormal sound generation site, a body site of the subject that is estimated to be close to the identified position.

Next, the output section 105 outputs abnormal site information which indicates the estimated abnormal sound generation site (step S7).

With this configuration, it is possible for the information processing system 100B to accurately measure the breathing signals of the subject W1 and identify an abnormal sound generation site associated with a disease of the subject W1. For example, by providing a medical expert or the like with the outputted breathing signals and abnormal sound generation site information, it is possible for the medical expert or the like to diagnose, with high accuracy, not only the health condition of the subject W1 but also in which body site of the subject W1 there is the possibility of occurrence of an anomaly.

### Example Display

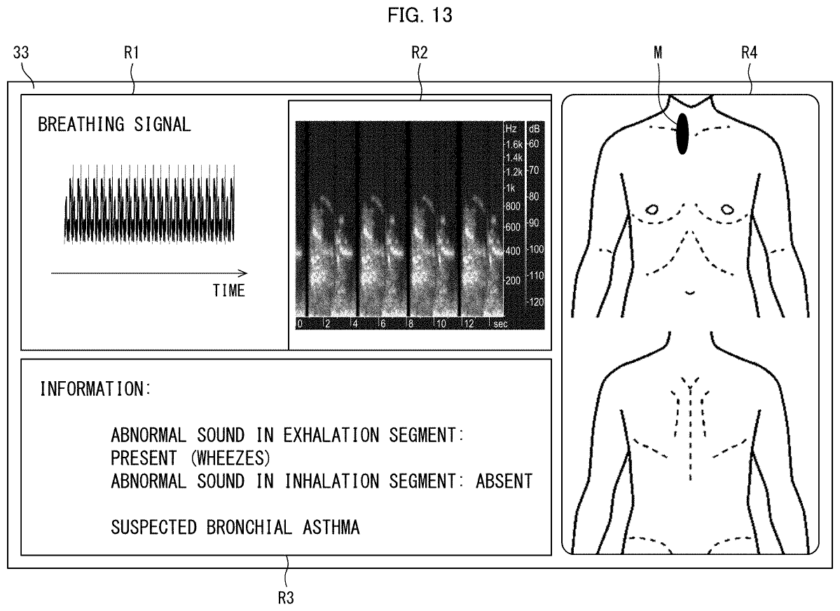

A screen display indicated on the display section 33 in a case where a sound associated with the breathing of the subject W1 contains an abnormal sound and the information processing apparatus 1B obtains (B1) to (B6) below from the information processing apparatus 1B is described here with use of FIG. 13.

(B1) An estimation result obtained by estimating in which breathing phase the abnormal sound is generated, the exhalation segment or the inhalation segment.

(B2) The breathing signals extracted from the detection signals.

(B3) The frequency characteristic of an abnormal sound signal indicating the abnormal sound.

(B4) An estimation result obtained by estimating an adventitious sound type.

(B5) Information regarding a health condition.

(B6) Abnormal site information.

FIG. 13 is a representation of an example of a display screen displayed on the display section 33 of the communication apparatus 3 of the information processing system 100B. As illustrated in FIG. 13, in the display section 33, in addition to the areas R1 to R3 illustrated in FIG. 9, an area R4 in which it is possible to display an abnormal site information of (B6) above regarding the subject may be further displayed. In the example illustrated in FIG. 13, an abnormal sound generation site is indicated by an oval mark M in the schematic views of the front and back sides of the body of the subject in the area R4.

For example, the medical expert or the like can refer to the display on the communication apparatus 3, to properly determine the health condition of the subject, the type and the site of the disease affecting the subject, and the need of medical intervention performed on the subject.

### Variation of Configuration of Information Processing System 100B

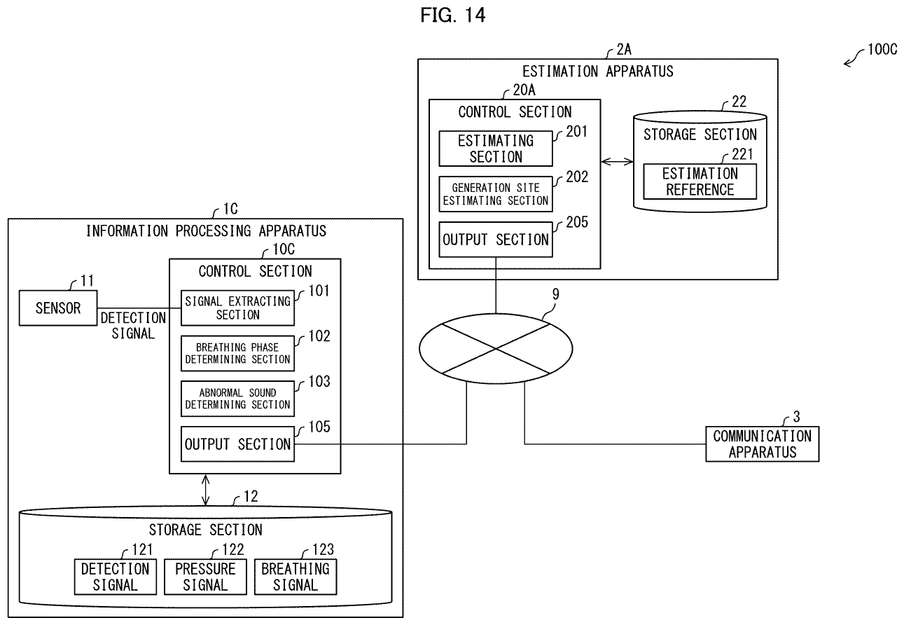

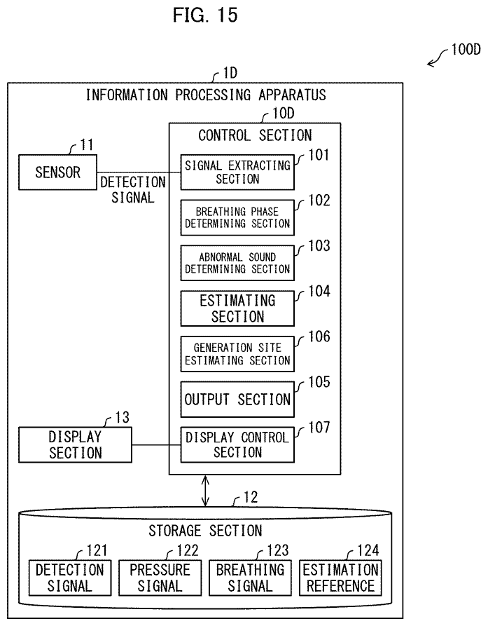

A variation of the configuration of the information processing system 100B is described here with use of FIGS. 14 and 15.

FIG. 14 is a functional block diagram illustrating a variation of the configuration of the information processing system. Compared with the information processing apparatus 1B illustrated in FIG. 11, an information processing system 100C illustrated in FIG. 14 differs from the information processing system 100B in that the estimating section 104 and the generation site estimating section 106, which are included in the control section 10B of the information processing apparatus 1B, are included respectively as an estimating section 201 and a generation site estimating section 202 in an estimation apparatus 2A, which is an external apparatus, not in a control section 10C of an information processing apparatus 1C. The estimation apparatus 2A includes a control section 20A and a storage section 22. Further, the control section 20A includes the estimating section 201 and an output section 205.

In accordance with the above variation, the output section 105 of the information processing apparatus 1C outputs the determination results provided by the breathing phase determining section 102 and the abnormal sound determining section 103 and breathing signals extracted from the respective area-specific detection signals, to the estimation apparatus 2A via the communication network 9.

The output section 205 of the estimation apparatus 2A outputs the estimation result provided by the estimating section 201, information regarding the health condition of the subject W1, and the estimation result provided by the generation site estimating section 202, to the communication apparatus 3 via the communication network 9.

FIG. 15 is a functional block diagram illustrating another variation of the configuration of the information processing system. An information processing system 100D illustrated in FIG. 15 includes, in an information processing apparatus 1D, all the components of the information processing system 100B illustrated in FIG. 11 and the information processing system 100C illustrated in FIG. 14. Further, the information processing apparatus 1D includes a display section 13, in addition to the sensor 11, the control section 10D, and the storage section 12.

The control section 10D includes a display control section 107 for displaying various types of information on the display section 13, in addition to the signal extracting section 101, the breathing phase determining section 102, the abnormal sound determining section 103, the estimating section 104, the output section 105, and the generation site estimating section 106. As an example, a display screen illustrated in FIG. 13 is displayed on the display section 13.

The information processing apparatus 1D may communicate with at least one communication apparatus 3 via the communication network 9. For example, information displayed on the display section 13 may also be displayed on the communication apparatuses 3 of respective medical experts W2, such as smartphones or tablet terminals.

The configurations of the information processing system 100C illustrated in FIG. 14 and the configuration of the information processing system 100D illustrated in FIG. 15 are merely examples, and the present disclosure is not limited thereto. Optionally, the sections of the information processing system may be provided as external apparatuses.

### Embodiment 3

The following description will discuss another embodiment (Embodiment 3) of the present disclosure. For convenience of description, a member having the same function as a member described in the above embodiment is assigned the same reference sign, and the description thereof is not repeated here.

### Outline of Information Processing System 100E

An information processing system 100E in accordance with Embodiment 3 of the present disclosure includes an abnormal sound determining section 103A for determining, based on a result obtained by performing an averaging process on a breathing signal which indicates a sound associated with the breathing of the subject W1, whether the sound associated with the breathing contains an abnormal sound.

### Configuration of Information Processing System 100E

The information processing system 100E has the same configuration as the above information processing systems 100 to 100D except that an abnormal sound determining section 103A is included instead of the abnormal sound determining section 103 of each of the information processing apparatuses 1 to 1D. In the following description, as the information processing system 100E, a system which has the same configuration as the information processing system 100 except that the abnormal sound determining section 103A is included instead of the abnormal sound determining section 103 of the information processing apparatus 1 is cited as an example.

### Processes Carried Out by Abnormal Sound Determining Section 103A

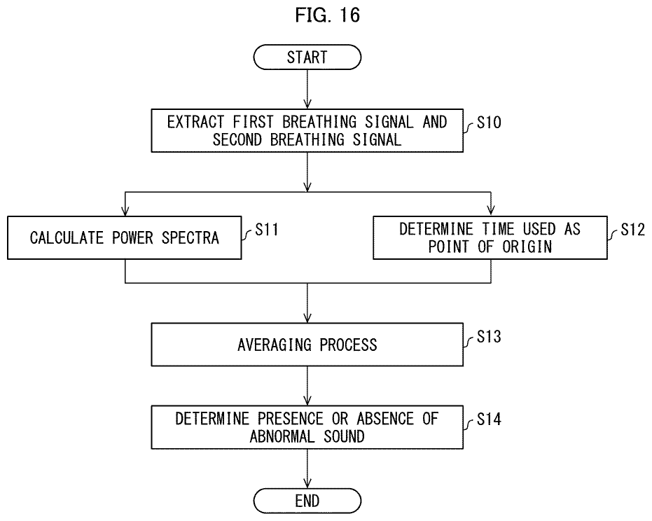

A flow of processes carried out by the abnormal sound determining section 103A is described here with use of FIG. 16. FIG. 16 is a flowchart illustrating an example flow of processes carried out by the abnormal sound determining section 103A.

First, the abnormal sound determining section 103A extracts, from a detection signal outputted from a sensor, a first breathing signal which has a frequency equal to or greater than a first frequency and a second breathing signal which has a frequency equal to or smaller than a second frequency (step S10). The first breathing signal and the second breathing signal only need to be breathing signals which do not contain a signal in the frequency band representing the heartbeat of the subject W1. That is, the first frequency only needs to be greater than the uppermost limit of the frequencies representing the heartbeat of the subject W1, and the second frequency only needs to be smaller than the lowermost limit of the frequencies representing the heartbeat of the subject W1. Typically, the frequencies representing heartbeats vary depending on age, lifestyle, etc. The first frequency and the second frequency can therefore preferably be set to any frequency according to the age and the lifestyle of the subject W1. For example, the first frequency may be set to not less than 3 Hz in a case where the subject W1 is a child, may be set to not less than 1.5 Hz in a case where the subject W1 is an athlete, and may be set to not less than 2.0 Hz in a case where the subject W1 is a general adult. The second frequency may be set to not more than 1 Hz regardless of the age or the lifestyle of the subject W1. This makes it possible to remove, from the breathing signal which represents a sound associated with the breathing of the subject W1, a signal of a frequency which represents the heartbeat, and therefore makes it possible to more accurately determine whether there is an abnormal sound.

The first frequency is preferably not less than 20 Hz in order to remove signals which represent vibrations or the like excluding sounds, and more preferably not less than 60 Hz in order to further remove signals which represent hum. A method for extracting the first breathing signal and the second breathing signal is not particularly limited, but in the present embodiment, a band-pass filter, a low-pass filter, etc. were used for the extraction of the first breathing signal and the second breathing signal. Although a high-pass filter can be used instead of a band-pass filter to extract the first breathing signal, a band-pass filter was adopted in the present embodiment for the prevention of aliasing caused in digital processing.

The first breathing signal and the second breathing signal may correspond to the breathing signal and the pressure signal extracted in the signal extracting section 101.

Next, the abnormal sound determining section 103A divides, by a predetermined time period, the first breathing signal extracted in step S10, and calculates a power spectrum for each of the divisions (step S11). As an example, the abnormal sound determining section 103A may divide the first breathing signal into unit segments each corresponding to an exhalation segment and an inhalation segment that are determined by the breathing phase determining section 102 or each corresponding to a breathing segment constituted by the exhalation segment and the inhalation segment, and calculate a power spectrum for each of the unit segments. Thus, the predetermined time period by which to divide the first breathing signal may be a time period of each unit segment, or may be arbitrarily-set time period. The arbitrarily-set time period is preferably not less than 0.1 seconds and not more than 0.8 seconds, and more preferably 0.1 seconds. That is, a plurality of power spectra may be calculated per unit segment. In the following description, the case of calculating a power spectrum of the first breathing signal unit segment by unit segment is cited as an example.

Next, the abnormal sound determining section 103A determines a time used as a point of origin in an averaging process (described later) performed on power spectra of the first breathing signal (step S12). As an example, the abnormal sound determining section 103A determines, from the second breathing signal extracted in step S10, for each of the unit segments, a time used as a point of origin in the averaging process performed on power spectra of the first breathing signal. In this determination, the abnormal sound determining section 103A may perform the process of removing, from the second breathing signal extracted in step S10, a large amplitude signal having an amplitude greater than a predetermined reference amplitude. For example, the time used as the point of origin may be determined from the second breathing signal having undergone the change of the amplitude of the large amplitude signal to the reference amplitude, or may be determined from the second breathing signal having undergone the removal of the large amplitude signal therefrom.

Next, the abnormal sound determining section 103A performs the averaging process on the power spectra calculated in step S11 (step S13). As an example, the abnormal sound determining section 103A aligns, with each other, the times used as points of origin for the respective unit segments determined in step S12, to perform averaging process on the power spectra of the first breathing signal of the respective unit segments. In this averaging process, the abnormal sound determining section 103A may perform a process for removing, from the first breathing signal, a large amplitude signal having an amplitude greater than a predetermined reference amplitude. For example, the averaging process may be performed on the power spectra of the first breathing signal having undergone the change of the amplitude of the large amplitude signal to the reference amplitude, or may be performed on the power spectra of the first breathing signal having undergone the removal of the large amplitude signal therefrom. In the averaging process, a method for averaging the power spectra of the first breathing signal is not particularly limited, but can arbitrarily be determined. Specific examples of the method for averaging the power spectra of the first breathing signal includes a power average for each frequency on the vertical axis (frequency axis) of the power spectra, the moving average of the power spectra, and a method (described below) in which a low-pass filter (low-pass IIR filter) is used.

Lastly, the abnormal sound determining section 103A determines, based on the result of the averaging process performed on the power spectra of the first breathing signal, whether the sound associated with the breathing of the subject W1 contains an abnormal sound (step S14). As an example, in a case where the ratio between the variance of the power spectra and the square of the power average of the first breathing signal is equal to or greater than a predetermined threshold, the abnormal sound determining section 103A determines that the sound associated with the breathing of the subject W1 contains an abnormal sound.

The abnormal sound determining section 103A may carry out a frequency-based correction process in addition to the above process. A frequency-based correction process is the process of temporal offset subtraction in each frequency of a sonogram which represents power spectra in three dimensions (frequency×amplitude×time). Specific examples of this process include the process of subtracting a value obtained by performing, for each frequency, time average taken over the displayed range of a sonogram and the process of removing an offset by applying, for each frequency, a high-pass filter on the temporal axis. By carrying out the frequency-based correction process, it is possible to remove the noise or the like having frequencies concentrated in specific frequencies such as those of hum noise and motor noise. This makes it possible to cause changes due to breathing to be obvious in a sonogram. This makes it easy to visually determine whether a sound associated with the breathing contains an abnormal sound.

Cited as an example in the present embodiment is the example in which each of the above-described processes (step S10 to S14) is carried out by the abnormal sound determining section 103A of the information processing apparatus 10E. However, the performer of each of the processes is not limited to the abnormal sound determining section 103A. For example, with the additional provision of an averaging processing section, step S10 may be carried out by the signal extracting section 101, step S12 may be carried out by the breathing phase determining section 102, steps S11 and S13 may be carried out by the averaging processing section, and step S14 may be carried out by the abnormal sound determining section 103A.

Each of the processes carried out by the abnormal sound determining section 103A is described here in more detail.

### Power Spectrum Calculation (Step S11)

The process of calculating power spectra of the first breathing signal is described here. Power spectra of the first breathing signal is calculated by performing the following processes.

(C1) Convert the first breathing signal into a sequence.

(C2) Prepare a signal set.

(C3) Assign a weight to each of the elements of the signal set with use of a window function.

(C4) Apply the Fourier transform to each of the elements of the signal set.

(C5) Calculate a power spectrum of each of the elements of the signal set.

(C6) Remove a singular value.

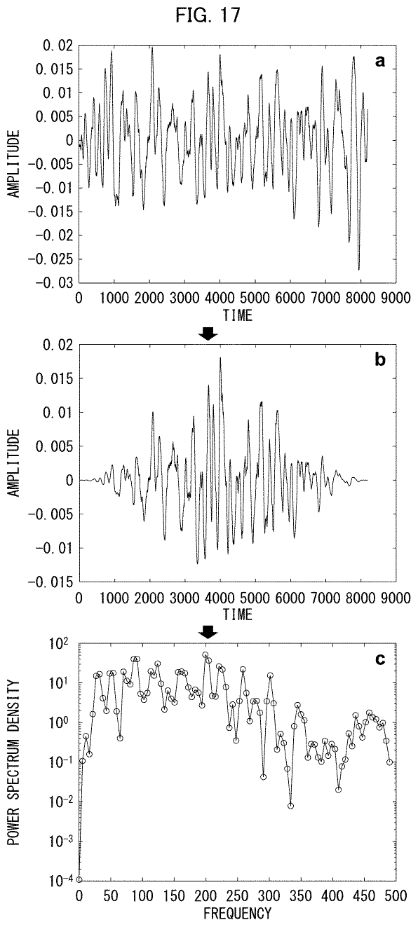

The processes (C1) to (C6) are described with reference to FIG. 17 by citing, as an example, the first breathing signal sampled at 44.1 kHz. FIG. 17 is a representation for explaining processes carried out for calculating power spectra of the first breathing signal.

First, the abnormal sound determining section 103A converts the first breathing signal sampled at 44.1 kHz, into a sequence y(n) (process (C1)).

Next, in the sequence representing the first breathing signal, a signal set is prepared (process (C2)). The signal set prepared in this process is, for example, a signal set obtained by shifting the starting point of the sequence y(n) by a predetermined number. For example, a signal set Y₈₁₉₂ in which the starting point of the sequence y(n) is shifted by a number of 2048 (approximately 46 msec) and which has 8192 elements (approximately 186 msec) is prepared.

Next, each of the elements of the signal set is assigned a weight with use of a window function (process (C3)). As an example, a signal set WY₈₁₉₂ is obtained by using a Hanning window function to assign a weight to each of the 8192 elements. When the process (3) is schematically expressed, a graph representing the first breathing signal as indicated in “a” of FIG. 17 is changed to a graph indicated in “b” of FIG. 17.

Next, the Fourier transform is applied to each of the elements having been assigned a weight with use of the window function (process (C4)). As an example, the Fourier transform is applied to the signal set WY₈₁₉₂, and CY₈₁₉₂ is obtained, accordingly. Here, CY₈₁₉₂ is complex data.

Next, the power spectrum of each of the elements after the Fourier transform is calculated (process (C5)). As an example, the square of the amplitude of each element of CY₈₁₉₂ is calculated, and a power spectrum P=pow(CY₈₁₉₂(n)) is obtained, accordingly. In FIG. 17, “c” indicates the power spectrum P=pow(CY₈₁₉₂(n)) of the first breathing signal.

Lastly, the process of removing a singular value from the power spectra of the first breathing signal may be carried out (process (6)). As an example, the singular value is a pulse noise.

The abnormal sound determining section 103A can carry out the processes (C1) to (C6), to calculate the power spectra of the first breathing signal having pulse noise removed therefrom.

### Calculation of Times Used as Points of Origin (Step S12)

The process of determining, from the second breathing signal, times used as points of origin in the averaging process of power spectra of the first breathing signal is described here. The times used as points of origin are determined by carrying out the following processes.

(D1) Convert the second breathing signal into a sequence.

(D2) Perform downsampling.

(D3) Perform digital filtering.

(D4) Prepare a filtered waveform which incorporates delay correction.

(D5) Calculate times used as points of origin.

The processes (D1) to (D5) are described here by citing, as an example, the second breathing signal sampled at 44.1 kHz.

First, the abnormal sound determining section 103A converts the second breathing signal sampled at 44.1 KHz, into a sequence x(n) (process (D1)).

Next, the abnormal sound determining section 103A performs downsampling to reduce the amount of data (process (D2)). As an example, the second breathing signal is sampled again at sampling intervals of N₂₀₄₈ (approximately 46 msec).

Next, digital filtering is applied to the sequence x(n) representing the downsampled second breathing signal (process D3). As an example, the IIR filter is applied to the sequence x(n).

Next, a filtered waveform incorporating the correction of the delay of the sequence x(n) which has undergone digital filtering and which represents the second breathing signal is prepared (process (D4)). Typically, applying a filter to a signal causes a delay. Applying the digital filtering of the process (D3) causes inconsistency between the sequence x(n) representing the second breathing signal and a sound representing actual breathing of the subject W1. In this process, a filtered waveform which incorporates the correction of this inconsistency is prepared.

Next, on the basis of the filtered waveform incorporating the delay correction, times used as points of origin in the averaging process performed on the power spectra of the first breathing signal are determined (process (D5)). The times used as points of origin may be any points in the breathing phases, but need to be in the same breathing phase, in the filtered waveform representing the second breathing signal. As such, in the present embodiment, a delay correction is made to the filtered waveform obtained by applying the IIR filter to the second breathing signal, and a time corresponding to an extremum in the waveform having undergone the delay correction is determined as the time used as a point of origin. Further, in the process D3, in a case of using an antisymmetric waveform obtained by applying the FIR filter to the second breathing signal, a time corresponding to the value at which the amplitude is 0 may be determined as the time used as a point of origin. Note that it is necessary to select between a point at which the amplitude changes from negative to positive and a point at which the amplitude changes from positive to negative.

### Specific Example

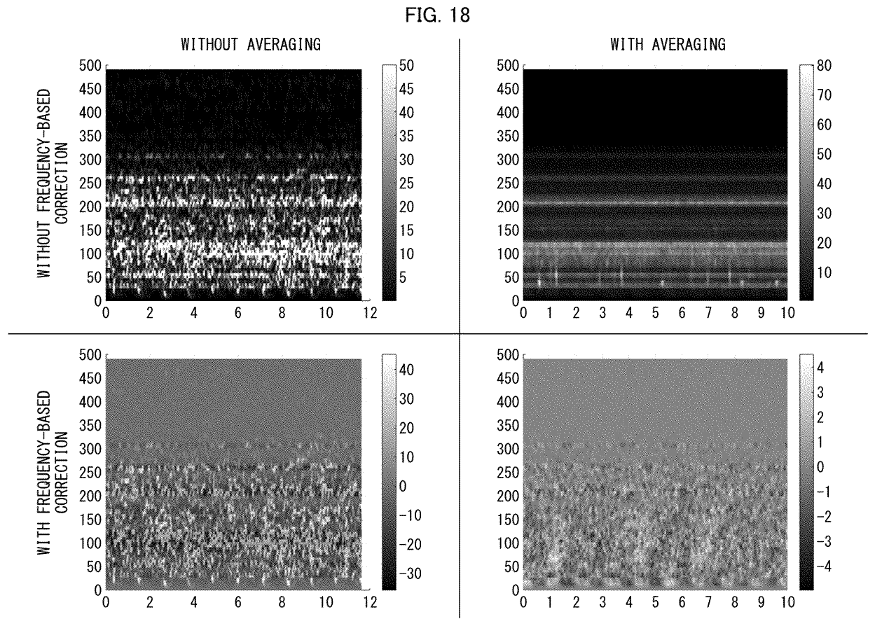

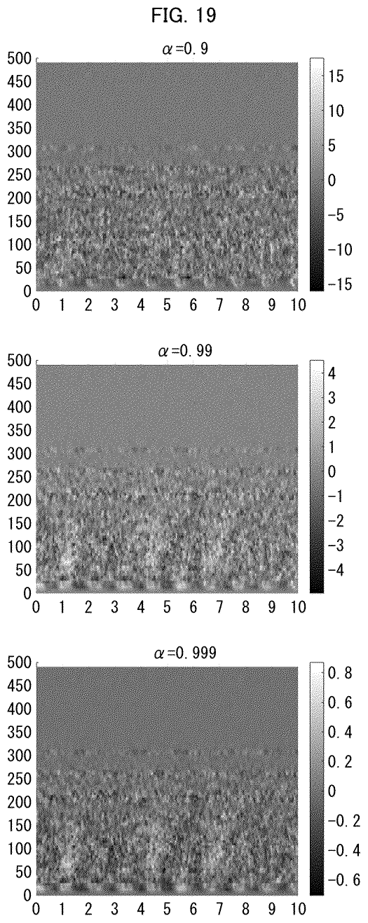

Specific examples of the power spectra of the first breathing signal and the power spectra of the first breathing signal having undergone the averaging process, the power spectra being outputted by the abnormal sound determining section 103A, are described here with use of FIGS. 18 and 19.

FIG. 18 is a spectrogram (sonogram) which represents the 100-th data to 350-th data of the power spectrum P=pow(CY₈₁₉₂(n)) of the first breathing signal described above. In the spectrogram, the vertical axis represents a power spectral density, the horizontal axis represents a frequency, and the light and shade represent an amplitude. As illustrated in FIG. 18, in a case of performing the averaging process and further performing the frequency-based correction (see the lower right graph of FIG. 18) on the power spectra of the first breathing signal, a band-shaped signal which appears to be in synchronization with the breathing of the subject W1 can be observed.

FIG. 19 is a representation of spectrograms obtained in a case of performing the averaging process on the power spectra of the first breathing signal. In FIG. 19, the filters used to perform the averaging process are first-order low-pass IIR filters designed with use of different averaging parameters α. The averaging parameter α is the pole of a first-order low-pass IIR filter. In other words, the parameter α represents the cutoff frequency of a first-order low-pass IIR filter, i.e., the point at which a transfer function becomes infinite. The value “0” of such an averaging parameter α indicates that no averaging has been performed, and the value “1” of the averaging parameter α indicates that averaging has been performed completely. As illustrated in FIG. 19, as the averaging parameter α approaches the value “1”, a band-shaped signal which appears to be in synchronization with the breathing of the subject W1 can be observed.

### Variation of Abnormal Sound Determining Section 103A

In the averaging process S12, the abnormal sound determining section 103A described above may carry out the process of linear combination of the power spectra of the first breathing signal which have undergone an n-th power mean operation (n is a natural number not less than 2), in addition to the averaging of the power spectra of the first breathing signal. As an example, the abnormal sound determining section 103A calculates, for each of the unit segments, the variance of the power spectrum of the first breathing signal, the variance being represented by <pow(w)²>-<pow(w)>², or the quantity of <pow(w)²>-2<pow(w)>².

### Calculation Method

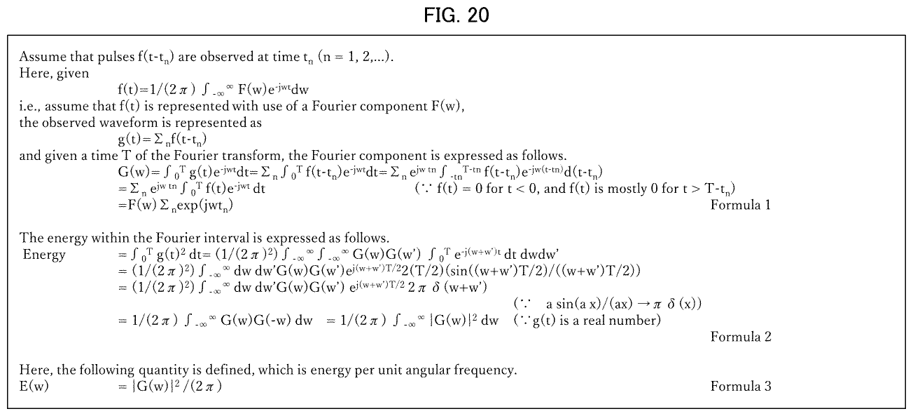

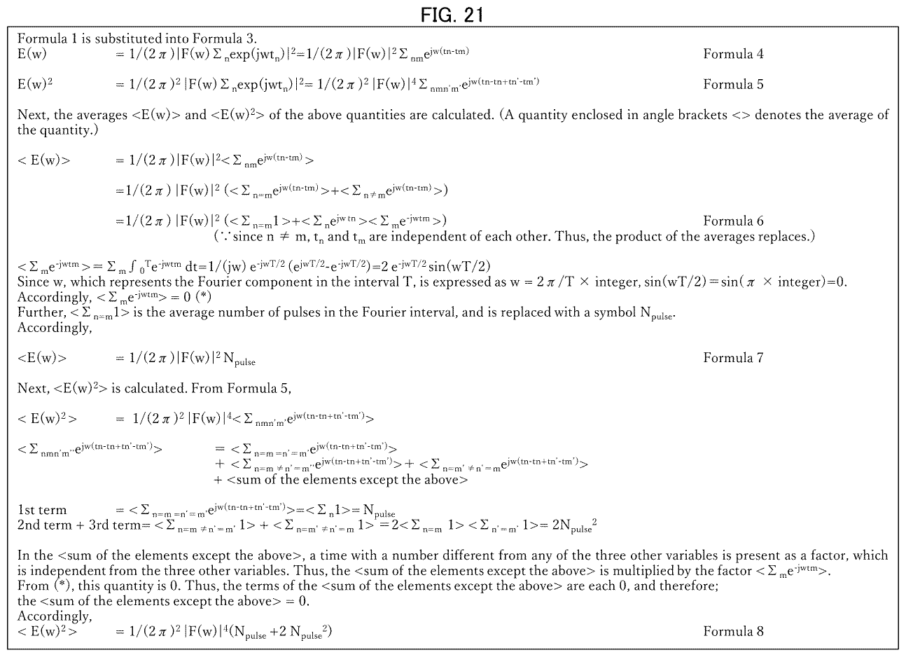

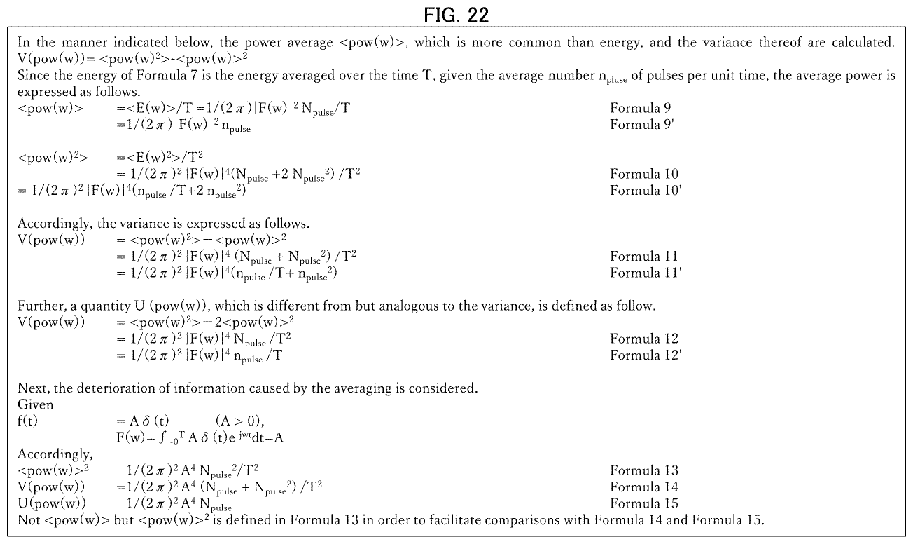

A method for calculating the value of <pow(w)²>-<pow(w)>² or <pow(w)²>-2<pow(w)>² is described here with use of FIGS. 20 to 22. FIGS. 20 to 22 illustrate a calculation method used on the assumption that the first breathing signal is a pulse sequence which is such that a plurality of pulses Af(t) which have the same waveform and which each have a pulse width of Δt are generated only during a time Te of a period Tr. Here, A is the amplitude of the pulse. The timing of the generation is a random timing during the time Te, and the density of the generation is n (pulse/sec) on average. Further, the stating time of Te is τₘ.

Assume that the first breathing signal is a pulse sequence of the pulses f(t−tₙ) observed at times tₙ (n=1, 2 . . . ). In this case, as illustrated in FIGS. 20 to 22, the value of <pow(w)²>-<pow(w)>² or <pow(w)²>-2<pow(w)>² is calculated in the following flow.

(E1) Perform the Fourier transform on the pulse sequence representing the first breathing signal (Formula 1).

(E2) Calculate energy in a Fourier interval (Formula 2 and Formula 3).

(E3) Calculate the average energy and the mean square energy in the Fourier interval (Formula 4 to Formula 8).

(E4) On the basis of the energy in the Fourier interval, calculate the power average and the mean square power of the power spectra (Formula 9 and Formula 10′).

(E5) Calculate the value of <pow(w)²>-<pow(w)>² or <pow(w)²>-2<pow(w)>² (Formula 11 to Formula 15).

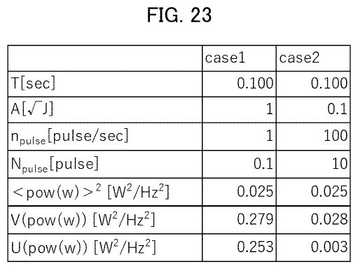

The results of calculations of the values of V(pow(w)) and U(pow(w)), which are <pow(w)²>-<pow(w)>² and <pow(w)²>-2<pow(w)>², respectively, of the power spectra of a breathing signal which represents fine crackles and a normal sound are described here with use of FIG. 23. FIG. 23 is a graph illustrating results of applying the above calculation method to models of fine crackles and a normal sound. In FIG. 23, casel indicates a case of a breathing signal which represents fine crackles and which contains a small number of pulses having large amplitudes, and case2 indicates a case of a breathing signal which represents a normal sound and which contains a great number of pulses having small amplitudes.

As illustrated in FIG. 23, the values of <pow(w)>² which represent the squares of the averages of the power spectra in case1 and case2 are the same. This means that with the averaging of power spectra of a breathing signal, it is difficult to distinguish between fine crackles and a normal sound. However, comparisons in the values of V(pow(w)) and U(pow(w)) between case1 and case2 show that the values of V(pow(w)) differ from each other approximately tenfold, and the values of U(pow(w)) differ from each other approximately hundredfold. Accordingly, the comparison between the values of V(pow(w)) or between the values of U(pow(w)) makes it possible to distinguish between fine crackles and a normal sound.

Thus, by comparing the value of <pow(w)²>-<pow(w)>² and the value of <pow(w)²>-2<pow(w)>² which are calculated from the power spectra of the first breathing signal respectively with the value of <pow(w)²>-<pow(w)>² and the value of <pow(w)²>-2<pow(w)>² which are calculated from power spectra of a breathing signal representing a normal sound, it is possible for the abnormal sound determining section 103A to more correctly determine whether a sound associated with the breathing of the subject W1 contains an abnormal sound.

### Variation of Information Processing System

The performer of each of the processes described in the above Embodiments is any performer, and is not limited to the above examples. For example, each of the steps (control method) illustrated in FIGS. 8 and 12 may be carried out by one or more information processing apparatuses. That is, the steps (control method) illustrated in FIGS. 8 and 12 may all be carried out by a single information processing apparatus, or may be shared among and carried out by a plurality of information processing apparatuses.

### Software Implementation Example

The functions of the information processing systems 100 and 100A to 100D (hereinafter, “systems”) and the information processing apparatuses 1 and 1A to 1D and the estimation apparatuses 2 and 2A (hereinafter, “apparatuses”) can be implemented by a program for causing a computer to function as the systems and the apparatuses, the program causing the computer to function as each of the control blocks (in particular, each of the sections of the control sections) of the systems and the information processing apparatus 1.

In this case, the systems and the apparatuses include a computer which has at least one control device (e.g. processor) and at least one storage device, which are hardware for carrying out the program. The program is executed via this control device and the storage device, so that each of the functions described in the above Embodiments is implemented.

The program may be recorded on one or more non-transitory computer-readable recording media. The recording media may be included in the above apparatuses, or may not be included in the above apparatuses. In the latter case, the program may be made available to the apparatuses via any wired or wireless transmission medium.

Further, some or all of the functions of the respective control blocks can be implemented by a logic circuit. For example, an integrated circuit in which the logic circuit that functions as each of the control block is formed is within the scope of the present invention. As another example, the functions of the respective control blocks can be implemented by a quantum computer.

Further, each of the processes described in the above Embodiments may be carried out by artificial intelligence (AI). In this case, the AI may operate in the control device, or may operate in another device (e.g. an edge computer or a cloud server).

### Additional Remark

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

As above, an information processing system in accordance with Aspect 1 of the present disclosure includes: a sensor for detecting, at a position where the sensor does not touch a subject or a position where the sensor touches the subject on a trunk of the subject, vibrations emitted from the trunk; a signal extracting section for extracting a pressure signal and a breathing signal from a detection signal outputted from the sensor, the pressure signal indicating a pressure on the sensor, the breathing signal indicating a sound associated with breathing of the subject; a breathing phase determining section for determining, based on a fluctuation in a strength of the pressure signal, an exhalation segment and an inhalation segment of the subject; an abnormal sound determining section for determining, based on the breathing signal, whether the sound associated with the breathing contains an abnormal sound; and an estimating section for estimating, in a case where the sound associated with the breathing contains an abnormal sound, in which segment the abnormal sound is generated, the exhalation segment or the inhalation segment.

In a case where there is some anomaly in the body of a subject, a sound associated with the breathing of the subject can contain an abnormal sound. It has been known that there are a plurality of types of abnormal sounds that can be contained in a sound associated with breathing. Thus, it can be possible to determine, from the type of abnormal sound, the type of anomaly occurring in the body of the subject.

Breathing has breathing phases, which are an inhalation segment in which air is taken into the lungs and an exhalation segment in which air is exhaled from the lungs. When it is possible to identify the breathing phase in which an abnormal sound is generated, it is possible to correctly identify what type the abnormal sound is.

With this configuration, the information processing system extracts a pressure signal and a breathing signal from a detection signal outputted from a sensor for detecting vibrations emitted from the trunk of a subject. The information processing system, based on the fluctuation in the strength of the pressure signal, determines the breathing phases (exhalation segment and inhalation segment) of a subject, and determines, based on the breathing signal, whether a sound associated with breathing contains an abnormal sound. In a case where the sound associated with breathing contains an abnormal sound, the information processing system estimates in which segment the abnormal sound is generated, the exhalation segment or the inhalation segment.

This enables the information processing system to accurately estimate, based on a detection signal outputted from a sensor for detecting vibrations emitted from the trunk of a subject, the breathing phase in which an abnormal sound contained in a sound associated with the breathing of the subject is generated. For example, it is possible for a medical expert to identify, based on the estimation result provided by the information processing system, what type the abnormal sound contained in the sound associated with the breathing of the subject is, and determine what type the anomaly occurring in the body of the subject is.

According to an information processing system of Aspect 2 of the present disclosure, in Aspect 1, the abnormal sound determining section may determine, in at least one of cases which are: a case where the breathing signal has a predetermined frequency characteristic; a case where a signal having a strength greater than a predetermined strength is contained for each frequency band of the breathing signal; a case where a signal having a strength greater than a predetermined strength is contained in a predetermined frequency band in the breathing signal; and a case where the breathing signal has a ratio between a strength of a signal in a frequency band detected during normal breathing and a strength of a signal in the predetermined frequency band, the ratio being equal to or greater than a predetermined threshold, that the sound associated with the breathing contains the abnormal sound.

With this configuration, the information processing system uses, for determining the presence or absence of an abnormal sound, at least one of the following regarding a subject: the frequency characteristic of a breathing signal; the strength of the breathing signal; and the ratio between the breathing signal during normal breathing and a signal in a predetermined frequency band. This enables the information processing system to correctly determine an abnormal sound contained in a sound associated with breathing.

According to an information processing system of Aspect 3 of the present disclosure, in Aspect 1 or 2, the abnormal sound includes an adventitious sound, and the estimating section may estimate, based on in which segment the abnormal sound is contained, the exhalation segment or the inhalation segment, what adventitious sound type the adventitious sound is.

With this configuration, an adventitious sound type is estimated based on in which segment an abnormal sound is contained, the exhalation segment or the inhalation segment. By considering not only the sound associated with breathing but also in which of the exhalation and inhalation segments an abnormal sound is generated, it is possible to accurately estimate an adventitious sound type.

According to an information processing system of Aspect 4 of the present disclosure, in Aspect 3, the estimating section may estimate the adventitious sound type, in accordance with a frequency of an abnormal sound signal which indicates the abnormal sound.

Different types of adventitious sounds have different frequency characteristics. With this configuration, it is possible to estimate, based on the frequency of an abnormal sound signal which indicates an abnormal sound, an adventitious sound type with higher reliability.

According to an information processing system of Aspect 5 of the present disclosure, in any one of Aspect 1 to Aspect 4, the estimating section is configured to: calculate a smoothed strength obtained by smoothing a fluctuation with time in strengths of signals in the same frequency band which contains the frequency of the abnormal sound; and estimate the adventitious sound type in accordance with the smoothed strength.

By performing a smoothing process on the strengths of signals in a predetermined frequency band which contains the frequency of an abnormal sound signal, it is possible to cancel out and reduce noise contained in the signal. With this configuration, it is possible for the information processing system to estimate an adventitious sound type with high accuracy and with high sensitivity.

According to an information processing system of Aspect 6 of the present disclosure, in any one of Aspect 3 to Aspect 5, the estimating section may estimate the adventitious sound type, in accordance with a change in a sound pressure of the abnormal sound and a time interval between occurrences of the abnormal sound, in the exhalation segment or the inhalation segment.

For example, the coarse crackles are lower-pitched sounds such as “rumbling” or “bubbling” sounds, and fine crackles are higher-pitched sounds such as “tearing up” sounds (crunch). Both the coarse crackles and the fine crackles have the characteristics of being discontinuous and generated in the inhalation segment. The rhonchi have the characteristics of being lower-pitched sounds such as “snoring” sounds, being continuous, and being generated in both the exhalation segment and the inhalation segment. The wheezes have the characteristic of being higher-pitched sounds (peep) such as “whistling” sounds, being continuous, and being generated in the exhalation segment. That is, adventitious sounds are different depending on their types, in terms of (1) in which of the exhalation and inhalation segments the adventitious sound is generated and (2) the time interval between occurrences of the adventitious sound (i.e. whether the adventitious sound is continuous or discontinuous).

With this configuration, the information processing system estimates an adventitious sound type in accordance with a change in the sound pressure of an abnormal sound and the time interval between occurrences of the abnormal sound, in the exhalation segment or the inhalation segment. This enables the information processing system to accurately estimate an adventitious sound type.

According to an information processing system of Aspect 7 of the present disclosure, in any one of Aspect 3 to Aspect 6, the information processing system may further include a health condition output section for outputting information regarding a health condition of the subject, the health condition being identified in accordance with the adventitious sound type estimated.

With this configuration, it is possible for the information processing system to output, for a subject, information (e.g. information on a disease or a lesion) regarding a health condition associated with the generation of an adventitious sound. For example, it is possible for a medical expert who refers to the outputted health information to early start performing a medical intervention on a subject.

According to an information processing system of Aspect 8 of the present disclosure, in any one of Aspect 1 to Aspect 7, the pressure signal may have a frequency of not less than 0.1 Hz and not more than 1 Hz.

With this configuration, it is possible to extract, based on a frequency characteristic, a pressure signal from a detection signal.

According to an information processing system of Aspect 9 of the present disclosure, in any one of Aspect 1 to Aspect 8, the breathing signal may have a frequency of not less than 20 Hz and not more than 1,000 Hz.

With this configuration, it is possible to extract, based on the frequency characteristic, a breathing signal from a detection signal.

According to an information processing system of Aspect 10 of the present disclosure, in any one of Aspect 1 to Aspect 9, the sensor for detecting the vibrations at a position where the sensor does not touch the subject may be installed on a piece of clothing the subject is wearing, at a position where the piece of clothing is sandwiched between the sensor and the trunk, or installed on a bed on which the subject is lying or a chair on which the subject is sitting, at a position where the trunk is supported.

With this configuration, it is possible for a sensor to detect vibrations emitted from the trunk of a subject, at a position where the sensor does not touch the subject or a position where the sensor touches the subject on the trunk. This means that the subject does not need to be in a predetermined detection area or to assume a predetermined position for detection. For example, in a case where a subject wearing clothes is in the state of reclining on a chair or lying down on a bed, the subject and the sensor are spontaneously close to each other, and it is therefore possible to accurately detect vibrations emitted from the trunk of the subject.

According to an information processing system of Aspect 11 of the present disclosure, in any one of Aspect 1 to Aspect 10, the trunk may include at least a chest of the subject.

With this configuration, it is possible to more accurately detect vibrations emitted from an organ of respiration of a subject.

According to an information processing system of Aspect 12 of the present disclosure, in any one of Aspect 1 to Aspect 11, the sensor may be thin plate-shaped.

With this configuration, it is possible to install the sensor at various locations. For example, it is possible to install the sensor on a piece of clothing a subject is wearing.

According to an information processing system of Aspect 13 of the present disclosure, in any one of Aspect 1 to Aspect 12, the sensor may include a piezoelectric sensor.

With this configuration, a reduction in thickness of the sensor is facilitated, and it is therefore possible to reduce the possibility of making the subject feel uncomfortable.

According to an information processing system of Aspect 14 of the present disclosure, in any one of Aspect 1 to Aspect 13, the sensor may include a plurality of detection areas which each output the detection signal, the signal extracting section may extract respective pressure signals and respective breathing signals from area-specific detection signals outputted from the plurality of detection areas, the respective pressure signals each being the pressure signal, the respective breathing signals each being the breathing signal.

With this configuration, the information processing system extracts respective pressure signals and respective breathing signals from area-specific detection signals outputted from a plurality of detection areas. This enables the information processing system to detect vibrations emitted from the trunk of a subject, in the detection area close to the location of generation of the vibrations. It is therefore possible for the information processing system to more accurately detect vibrations emitted from the trunk of a subject.

According to an information processing system of Aspect 15 of the present disclosure, in Aspect 14, the information processing system may further include a site estimating section for estimating, based on the respective breathing signals extracted from the area-specific detection signals, an abnormal sound generation site inside a body of the subject.

With this configuration, the information processing system estimates, based on respective breathing signals extracted from the area-specific detection signals, an abnormal sound generation site inside the body of a subject. This makes it possible to estimate in what site of the body of a subject there is the possibility of occurrence of an anomaly (e.g. inflammation).

According to an information processing system of Aspect 16 of the present disclosure, in any one of Aspect 3 to Aspect 7, the signal extracting section may use a band-pass filter corresponding to a frequency in accordance with the adventitious sound type to extract the breathing signal from the detection signal.

Since adventitious sounds have distinct frequency characteristics depending on their types, using band-pass filters corresponding to the respective frequencies of types of adventitious sounds makes it possible to extract a breathing signal from a detection signal.

An information processing apparatus of Aspect 17 of the present disclosure includes a signal extracting section for extracting a pressure signal and a breathing signal from a detection signal outputted from a sensor for detecting, at a position where the sensor does not touch a subject or a position where the sensor touches the subject on a trunk of the subject, vibrations emitted from the trunk, the pressure signal indicating a pressure on the sensor, the breathing signal indicating a sound associated with breathing of the subject; a breathing phase determining section for determining, based on a fluctuation in a strength of the pressure signal, an exhalation segment and an inhalation segment of the subject; an abnormal sound determining section for determining, based on the breathing signal, whether the sound associated with the breathing contains an abnormal sound; and an estimating section for estimating, in a case where the sound associated with the breathing contains an abnormal sound, in which segment the abnormal sound is generated, the exhalation segment or the inhalation segment. This configuration provides the same effect that is provided by the information processing system of Aspect 1.

An information processing apparatus of Aspect 18 of the present disclosure includes a signal extracting section for extracting a pressure signal and a breathing signal from a detection signal outputted from a sensor for detecting, at a position where the sensor does not touch a subject or a position where the sensor touches the subject on a trunk of the subject, vibrations emitted from the trunk, the pressure signal indicating a pressure on the sensor, the breathing signal indicating a sound associated with breathing of the subject; a breathing phase determining section for determining, based on a fluctuation in a strength of the pressure signal, an exhalation segment and an inhalation segment of the subject; and an abnormal sound determining section configured to determine, based on the breathing signal, whether the sound associated with the breathing contains an abnormal sound, and further includes an output section for outputting, in a case where the sound associated with the breathing contains an abnormal sound, a determination result provided by the breathing phase determining section and a determination result provided by the abnormal sound determining section, to an external apparatus which includes an estimating section for estimating in which segment the abnormal sound is generated, the exhalation e inhalation segment. This configuration provides the same effect that is provided by the information processing system of Aspect 1.

A control method of Aspect 19 of the present disclosure is a control method carried out by one or more information processing apparatuses, and the control method includes: a signal extracting step of extracting a pressure signal and a breathing signal from a detection signal outputted from a sensor for detecting, at a position where the sensor does not touch a subject or a position where the sensor touches the subject on a trunk of the subject, vibrations emitted from the trunk, the pressure signal indicating a pressure on the sensor, the breathing signal indicating a sound associated with breathing of the subject; a breathing phase determining step of determining, based on a fluctuation in a strength of the pressure signal, an exhalation segment and an inhalation segment of the subject; an abnormal sound determining step of determining, based on the breathing signal, whether the sound associated with the breathing contains an abnormal sound; and an estimating step of estimating, in a case where the sound associated with the breathing contains an abnormal sound, in which segment the abnormal sound is generated, the exhalation segment or the inhalation segment. This configuration provides the same effect that is provided by the information processing system of Aspect 1.

A program of Aspect 20 of the present disclosure is a program for controlling a computer to cause the computer to serve as the information processing system of Aspect 1, the program causing the computer to function as the signal extracting section, the breathing phase determining section, the abnormal sound determining section, and the estimating section. This configuration provides the same effect that is provided by the information processing system of Aspect 1.

According to an information processing system of Aspect 21 of the present disclosure, in any one of Aspect 1 to Aspect 18, the abnormal sound determining section is configured to: calculate power spectra of a first breathing signal which has a frequency equal to or greater than a first frequency and which is contained in the detection signal, for unit segments each corresponding to each of the exhalation segment and the inhalation segment or each corresponding to a breathing segment constituted by the exhalation segment and the inhalation segment; determine, from a second breathing signal which has a frequency equal to or smaller than a second frequency and which is contained in the breathing signal, individually for the unit segments, times used as points of origin in an averaging process performed on the power spectra; and determine, based on a result of the averaging process performed with the points of origin of the power spectra in the unit segments aligned with each other, whether the sound associated with the breathing contains an abnormal sound.

With this configuration, the averaging process is carried out after a signal in an unnecessary frequency band is removed from the breathing signal, and it is therefore possible to more accurately determine whether a sound associated with the breathing of a subject contains an abnormal sound.

According to an information processing system of Aspect 22 of the present disclosure, in Aspect 21, in the averaging process, in a case where a ratio between a variance of the power spectra and a square of a power average of the first breathing signal is equal to or greater than a predetermined threshold, the abnormal sound determining section is configured to determine that the sound associated with the breathing contains an abnormal sound.

With this configuration, it is possible to accurately determine whether an abnormal sound is contained and distinguish between abnormal sounds similar to each other.

According to an information processing system of Aspect 23 of the present disclosure, in Aspect 21, in a case where at least one of signals which are the first breathing signal and the second breathing signal contains a large amplitude signal having an amplitude greater than a predetermined reference amplitude, the averaging process is performed with the amplitude of the large amplitude signal changed to the predetermined reference amplitude.

With this configuration, it is possible to simplify a complicated process. This makes it possible to determine whether a sound associated with the breathing of a subject contains an abnormal sound not only accurately but also efficiently.

According to an information processing system of Aspect 24 of the present disclosure, in Aspect 21, in a case where at least one of signals which are the first breathing signal and the second breathing signal contains a large amplitude signal having an amplitude greater than a predetermined reference amplitude, the averaging process is performed with the large amplitude signal removed in the unit segments.

With this configuration, it is possible to simplify a complicated process. This makes it possible to determine whether a sound associated with the breathing of a subject contains an abnormal sound not only accurately but also efficiently.

According to an information processing system of Aspect 25 of the present disclosure, in Aspect 21, based on a value calculated by linear combination of the power spectra having undergone an n-th power mean operation (n is a natural number not less than 2), whether the sound associated with the breathing contains an abnormal sound is determined.

With this configuration, it is possible to make a highly accurate determination even in a case of a breathing signal that cannot be distinguished from a normal sound via the averaging process.

### REFERENCE SIGNS LIST

1, 1A, 1B, 1C, 1D: Information processing apparatus
2, 2A: Estimation apparatus
3: Communication apparatus
11: Sensor
100, 100A, 100B, 100C, 100D: Information processing system
101: Signal extracting section
102: Breathing phase determining section
103: Abnormal sound determining section
104, 201: Estimating section
105, 205: Output section (health condition output section)
106, 202: Generation site estimating section

1. An information processing system, comprising:
a sensor for detecting, at a position where the sensor does not touch a subject or a position where the sensor touches the subject on a trunk of the subject, vibrations emitted from the trunk;
a signal extracting section for extracting a pressure signal and a breathing signal by using frequency separation from a detection signal outputted from the sensor, the pressure signal indicating a pressure on the sensor, the breathing signal indicating a sound associated with breathing of the subject;
a breathing phase determining section for determining, based on a fluctuation in a strength of the pressure signal, an exhalation segment and an inhalation segment of the subject;
an abnormal sound determining section for determining, based on the breathing signal, whether the sound associated with the breathing contains an abnormal sound; and
an estimating section for estimating, in a case where the sound associated with the breathing contains an abnormal sound, in which segment the abnormal sound is generated, the exhalation segment or the inhalation segment.

2. The information processing system according to claim 1, wherein
the abnormal sound determining section is configured to determine, in at least one of cases which are:
a case where the breathing signal has a predetermined frequency characteristic;
a case where a signal having a strength greater than a predetermined strength is contained for each frequency band of the breathing signal;
a case where a signal having a strength greater than a predetermined strength is contained in a predetermined frequency band in the breathing signal; and
a case where the breathing signal has a ratio between a strength of a signal in a frequency band detected during normal breathing and a strength of a signal in the predetermined frequency band, the ratio being equal to or greater than a predetermined threshold, that the sound associated with the breathing contains the abnormal sound.

3. The information processing system according to claim 1, wherein
the abnormal sound includes an adventitious sound, and
the estimating section is configured to estimate, based on in which segment the abnormal sound is contained, the exhalation segment or the inhalation segment, what adventitious sound type the adventitious sound is.

4. The information processing system according to claim 3, wherein
the estimating section is configured to estimate the adventitious sound type, in accordance with a frequency of an abnormal sound signal which indicates the abnormal sound.

5. The information processing system according to claim 4, wherein
the estimating section is configured to:
calculate a smoothed strength obtained by smoothing a fluctuation with time in strengths of signals in the same frequency band which contains the frequency of the abnormal sound; and
estimate the adventitious sound type in accordance with the smoothed strength.

6. The information processing system according to claim 3, wherein
the estimating section is configured to estimate the adventitious sound type, in accordance with a change in a sound pressure of the abnormal sound and a time interval between occurrences of the abnormal sound, in the exhalation segment or the inhalation segment.

7. The information processing system according to claim 3, further comprising
a health condition output section for outputting information regarding a health condition of the subject, the health condition being identified in accordance with the adventitious sound type estimated.

8. The information processing system according to claim 1, wherein
the pressure signal has a frequency of not less than 0.1 Hz and not more than 1 Hz, and
the breathing signal has a frequency of not less than 20 Hz and not more than 1,000 Hz.

9. (canceled)

10. The information processing system according to claim 1, wherein
the sensor for detecting the vibrations at a position where the sensor does not touch the subject is
installed on a piece of clothing the subject is wearing, at a position where the piece of clothing is sandwiched between the sensor and the trunk, or
installed on a bed on which the subject is lying or a chair on which the subject is sitting, at a position where the trunk is supported.

11. The information processing system according to claim 1, wherein
the trunk includes at least a chest of the subject.

12. The information processing system according to claim 1, wherein
the sensor is thin plate-shaped and includes a piezoelectric sensor.

13. (canceled)

14. The information processing system according to claim 1, wherein
the sensor includes a plurality of detection areas which each output the detection signal,
the signal extracting section is configured to extract respective pressure signals and respective breathing signals from area-specific detection signals outputted from the plurality of detection areas, the respective pressure signals each being the pressure signal, the respective breathing signals each being the breathing signal.

15. The information processing system according to claim 14, further comprising
a generation site estimating section for estimating, based on the respective breathing signals extracted from the area-specific detection signals, an abnormal sound generation site inside a body of the subject.

16. The information processing system according to claim 3, wherein
the signal extracting section is configured to use a band-pass filter corresponding to a frequency in accordance with the adventitious sound type to extract the breathing signal from the detection signal.

17-18. (canceled)

19. A control method carried out by one or more information processing apparatuses, the control method comprising:
a signal extracting step of extracting a pressure signal and a breathing signal by using frequency separation from a detection signal outputted from a sensor for detecting, at a position where the sensor does not touch a subject or a position where the sensor touches the subject on a trunk of the subject, vibrations emitted from the trunk, the pressure signal indicating a pressure on the sensor, the breathing signal indicating a sound associated with breathing of the subject;
a breathing phase determining step of determining, based on a fluctuation in a strength of the pressure signal, an exhalation segment and an inhalation segment of the subject;
an abnormal sound determining step of determining, based on the breathing signal, whether the sound associated with the breathing contains an abnormal sound; and
an estimating step of estimating, in a case where the sound associated with the breathing contains an abnormal sound, in which segment the abnormal sound is generated, the exhalation segment or the inhalation segment.

20. (canceled)

21. The information processing system according to claim 1, wherein
the abnormal sound determining section is configured to:
calculate power spectra of a first breathing signal which has a frequency equal to or greater than a first frequency and which is contained in the detection signal, for unit segments each corresponding to each of the exhalation segment and the inhalation segment or each corresponding to a breathing segment constituted by the exhalation segment and the inhalation segment;
determine, from a second breathing signal which has a frequency equal to or smaller than a second frequency and which is contained in the breathing signal, individually for the unit segments, times used as points of origin in an averaging process performed on the power spectra; and
determine, based on a result of the averaging process performed with the points of origin of the power spectra in the unit segments aligned with each other, whether the sound associated with the breathing contains an abnormal sound.

22. The information processing system according to claim 21, wherein
in the averaging process, in a case where a ratio between a variance of the power spectra and a square of a power average of the first breathing signal is equal to or greater than a predetermined threshold, the abnormal sound determining section is configured to determine that the sound associated with the breathing contains an abnormal sound.

23. The information processing system according to claim 21, wherein
in a case where at least one of signals which are the first breathing signal and the second breathing signal contains a large amplitude signal having an amplitude greater than a predetermined reference amplitude, the averaging process is performed with the amplitude of the large amplitude signal changed to the predetermined reference amplitude.

24. The information processing system according to claim 21, wherein
in a case where at least one of signals which are the first breathing signal and the second breathing signal contains a large amplitude signal having an amplitude greater than a predetermined reference amplitude, the averaging process is performed with the large amplitude signal removed.

25. The information processing system according to claim 21, wherein
based on a value calculated by linear combination of the power spectra having undergone an n-th power mean operation (n is a natural number not less than 2), whether the sound associated with the breathing contains an abnormal sound is determined.
